(19) United States
(12) Patent Application Publication
(10) Pub. No.: US 2009/0247590 A1
(43) Pub. Date: Oct. 1, 2009

(54) Indane amides

(75) Inventors: Yamin Wang, Sandy Hook, CT (US); Tindy Li, West Haven, CT (US); Eric Mull, Guilford, CT (US)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Pharmaceuticlas Corporation, West Haven, CT (US)

(21) Appl. No.: 11/665,407

(22) PCT Filed: Oct. 14, 2005
(86) PCT No.: PCT/US05/37209

§ 371 Date: Apr. 13, 2007

Related U.S. Application Data
(60) Provisional application No. 60/619,072, filed on Oct. 15, 2004.

(51) Int. Cl. A61K 31/404; C07D 209/14; C07D 213/36; A61K 31/4406; A61P 35/00

(52) U.S. Cl. 514/357; 548/507; 546/332; 514/415

(57) ABSTRACT

The invention relates to novel compounds and processes for their preparation and their use for preparing medicaments for the treatment of disorders, especially hyper-proliferative disorders.

This application claims benefit of U.S. Provisional Application Ser. No. 60/619,072; filed on Oct. 15, 2004, the content of which is incorporated herein by reference in its entirety.

The invention relates to novel compounds and processes for their preparation and their use for preparing medicaments for the treatment of disorders, especially hyper-proliferative disorders.

Various benzamides have been disclosed in WO 2003/092688 (AstraZeneca), WO 2003/087057 (AstraZeneca), US2004/0142953 (MethylGene), WO 2002/069947 (MethylGene), WO 2003/024448 (MethylGene), WO 2004/069823 (MethylGene), WO 2004/035525 (MethylGene), WO 2004/052838 (Roche), WO 2004/069803 (Roche), JP 2003/137866 (Sankyo), JP 11302173 (Mitsui) and WO 2004/058234 (Schering AG) as anti-proliferative agents.

In one embodiment, the present invention provides a compound of formula (I)

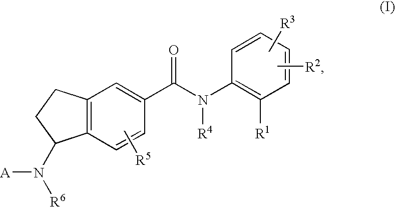

wherein
A represents

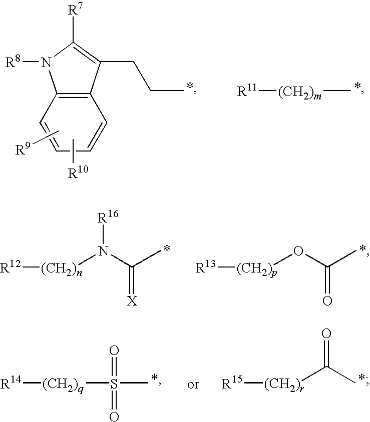

m, n, p, q and r represent 0, 1, 2, or 3;
R¹ represents hydroxy, alkoxy, amino or alkylamino;
R² represents hydrogen, alkyl or halo;
R³ represents hydrogen, alkyl or halo;
R⁴ represents hydrogen or alkyl;
R⁵ represents hydrogen, alkyl or halo;
R⁶ represents hydrogen; or
R⁶ represents alkyl, wherein alkyl can be substituted with 0, 1 or 2 substituents selected from the group consisting of halo, hydroxy, alkoxy, amino and alkylamino; or
R⁶ represents alkylcarbonyl; or
R⁶ represents alkylaminocarbonyl; or
R⁶ represents alkylsulfonyl;
R⁷ represents hydrogen or alkyl;
R⁸ represents hydrogen or alkyl;
R⁹ represents hydrogen, alkyl, halo, hydroxy or alkoxy;
R¹⁰ represents hydrogen, alkyl, halo, hydroxy or alkoxy;
R¹¹ represents hydrogen, phenyl, or benzthiazolyl;
R¹² represents pyridyl, thiazolyl, or indolyl optionally substituted with 1 or 2 substituents independently selected from the group consisting of alkyl, alkoxy and halo; or
R¹² represents phenyl optionally substituted with 1 or 2 substituents independently selected from the group consisting of alkyl, alkoxy, halo and amino;
R¹³ represents pyridyl or phenyl optionally substituted with 1 or 2 substituents independently selected from the group consisting of alkyl, alkoxy and halo;
R¹⁴ represents alkyl or phenyl optionally substituted with 1 or 2 substituents independently selected from the group consisting of alkyl, alkoxy and halo;
R¹⁵ represents hydrogen, pyridyl, pyridyloxy, phenoxy, or phenyl optionally substituted with 1 or 2 substituents independently selected from the group consisting of alkyl, alkoxy and halo;
R¹⁶ represents hydrogen or alkyl;
X represents oxygen or sulfur;
or a pharmaceutically acceptable salt thereof.

The compounds of this invention may contain one or more asymmetric centers, depending upon the location and nature of the various substituents desired. Asymmetric carbon atoms may be present in the (R) or (S) configuration. In certain instances, asymmetry may also be present due to restricted rotation about a given bond, for example, the central bond adjoining two substituted aromatic rings of the specified compounds. Substituents on a ring may also be present in either cis or trans form, and a substituent on a double bond may be present in either =Z- or =E-form. It is intended that all such configurations (including enantiomers and diastereomers) are included within the scope of the present invention. Preferred compounds are those with the absolute configuration of the compound of this invention which produces the more desirable biological activity. Separated, pure or partially purified isomers or racemic mixtures of the compounds of this invention are also included within the scope of the present invention. The purification of said isomers and the separation of said isomeric mixtures can be accomplished by standard techniques known in the art.

For the compounds containing one or more asymmetric centers, (±), (+), or (−) is used to describe the racemic mixture, the enantiomer with the positive optical rotation, or the negative rotation, respectively. In the absence of any (+) or (−) sign before a structure or a chemical name, the compound described is a racemic mixture with the relative stereochemistry shown.

The invention also relates to tautomers of the compounds, depending on the structure of the compounds.

Salts for the purposes of the invention are preferably pharmaceutically acceptable salts of the compounds according to the invention.

Pharmaceutically acceptable salts of the compounds (I) include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid and benzoic acid.

Pharmaceutically acceptable salts of the compounds (I) also include salts of customary bases, such as for example and preferably alkali metal salts (for example sodium and potassium salts, alkaline earth metal salts (for example calcium and magnesium salts) and ammonium salts derived from ammonia or organic amines having 1 to 16 carbon atoms, such as illustratively and preferably ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

Solvates for the purposes of the invention are those forms of the compounds that coordinate with solvent molecules to form a complex in the solid or liquid state. Hydrates are a specific form of solvates, where the solvent is water.

For the purposes of the present invention, the substituents have the following meanings, unless otherwise specified:

Alkyl represents a linear or branched alkyl radical having generally 1 to 6, or, in another embodiment, 1 to 4, or in yet another embodiment 1 to 3 carbon atoms, illustratively representing methyl, ethyl, n-propyl, isopropyl, tert-butyl, n-pentyl and n-hexyl.

Alkoxy represents a straight-chain or branched hydrocarbon radical having 1 to 6, or, in another embodiment, 1 to 4, or in yet another embodiment 1 to 3 carbon atoms and bound via an oxygen atom, illustratively representing methoxy, ethoxy, propoxy, isopropoxy, butoxy, isobutoxy, pentoxy, isopentoxy, hexoxy, isohexoxy. The terms “alkoxy” and “alkyloxy” are often used synonymously.

Alkylamino represents an alkylamino radical having one or two (independently selected) alkyl substituents, illustratively representing methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Alkylaminocarbonyl represents an alkylaminocarbonyl radical having one or two (independently selected) alkyl substituents, illustratively representing methylaminocarbonyl, ethylaminocarbonyl, n-propylaminocarbonyl, isopropylamino-carbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylamino-carbonyl and N-n-hexyl-N-methylaminocarbonyl.

Alkylcarbonyl represents an carbonyl radical having one alkyl substituent, illustratively representing methylcarbonyl or ethylcarbonyl.

Alkylsulfonyl represents *-S(O)₂alkyl, illustratively representing methylsulfonyl or ethylsulfonyl.

Halo represents fluorine, chlorine, bromine or iodine.

A * symbol next to a bond or a [symbol] line through a bond denotes the point of attachment in the molecule.

In another embodiment, except for intermediates, chemically unstable compounds are excluded in the context of the present invention. For example, a chemically unstable compound would be one where two nitrogen or oxygen substituents are bonded to a single aliphatic carbon atom. Another example of a chemically unstable compound would be one where an alkoxy group is bonded to the unsaturated carbon of an alkene to form an enol ether. Furthermore, an aliphatic carbon atom attached to oxygen may not also bear a chloro, bromo or iodo substituent, and when any alkyl group is attached to O, S, or N, and bears a hydroxyl substituent, then the hydroxyl substituent is separated by at least two carbon atoms from the O, S, or N to which the alkyl group is attached.

In another embodiment, the present invention provides a compound of formula (I), wherein A represents

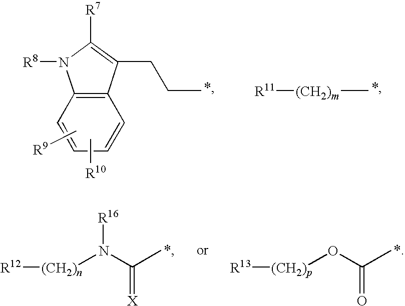

In another embodiment, the present invention provides a compound of formula (I), wherein A represents

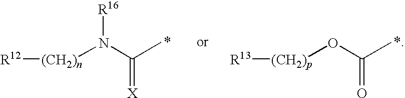

In another embodiment, the present invention provides a compound of formula (I), wherein

R¹ represents hydroxy or amino;
R² represents hydrogen;
R³ represents hydrogen;
R⁴ represents hydrogen;
R⁵ represents hydrogen;
R⁶ represents hydrogen; or
R⁶ represents alkyl.
In yet another embodiment, the present invention provides a compound of formula (II)

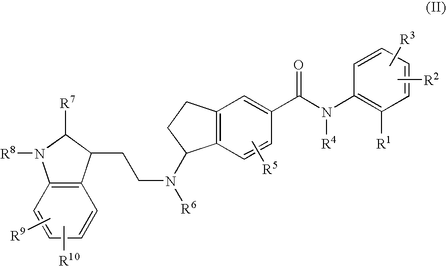

wherein
R¹ represents hydroxy, alkoxy, amino or alkylamino;
R² represents hydrogen, alkyl or halo;
R³ represents hydrogen, alkyl or halo;
R⁴ represents hydrogen or alkyl;
R⁵ represents hydrogen, alkyl or halo;
R⁶ represents hydrogen; or
R⁶ represents alkyl, wherein alkyl can be substituted with 0, 1 or 2 substituents selected from the group consisting of halo, hydroxy, alkoxy, amino and alkylamino; or
R⁶ represents alkylcarbonyl; or
R⁶ represents alkylaminocarbonyl; or
R⁶ represents alkylsulfonyl;
R⁷ represents hydrogen, alkyl, methoxymethyl or methoxyethyl;
R⁸ represents hydrogen or alkyl;
R⁹ represents hydrogen, alkyl, halo, hydroxy or alkoxy;
R¹⁰ represents hydrogen, alkyl, halo, hydroxy or alkoxy;
or a pharmaceutically acceptable salt, solvate, or a solvate of a salt thereof.

In yet another embodiment, the present invention provides a compound of formula (II), wherein

R¹ represents hydroxy or amino;
R⁶ represents hydrogen, alkyl or halo;
R³ represents hydrogen;
R⁴ represents hydrogen;
R⁵ represents hydrogen;
R⁶ represents hydrogen; or
R⁶ represents alkyl, wherein alkyl can be substituted with 0, 1 or 2 substituents selected from the group consisting of halo, hydroxy, alkoxy, amino and alkylamino; or
R⁶ represents alkylcarbonyl; or
R⁶ represents alkylaminocarbonyl; or
R⁶ represents alkylsulfonyl;
R⁷ represents hydrogen;
R⁸ represents hydrogen;
R⁹ represents hydrogen;
R¹⁰ represents hydrogen;
or a pharmaceutically acceptable salt, solvate, or a solvate of a salt thereof.

In another embodiment, the present invention provides a compound of formula (II), wherein R⁶ is not hydrogen.

In another embodiment, the present invention provides a compound of formula (II), wherein R¹ is alkylamino having one alkyl substituent.

In another embodiment, the present invention provides a compound of formula (II), wherein R¹ is amino.

In general, the compounds used in this invention may be prepared by standard techniques known in the art, by known processes analogous thereto, and/or by the processes described herein, using starting materials which are either commercially available or producible according to routine, conventional chemical methods. The particular process to be utilized in the preparation of the compounds of this invention depends upon the specific compound desired. Such factors as whether the amine is substituted or not, the selection of the specific substituents possible at various locations on the molecule, and the like, each play a role in the path to be followed in the preparation of the specific compounds of this invention. Those factors are readily recognized by one of ordinary skill in the art.

The general synthesis of a compound of this invention is described below in Flow Diagrams I-IV. The starting materials and/or intermediates are either commercially available or are prepared in similar manner as described in the literature procedures or the procedures described in the specific examples.

The right-hand portion of the compounds of Formula (I), the optionally substituted N-phenylacrylamide moiety, may be constructed by forming connection A, or connections A and B, described further below. The left-hand portion may be constructed by forming connection C.

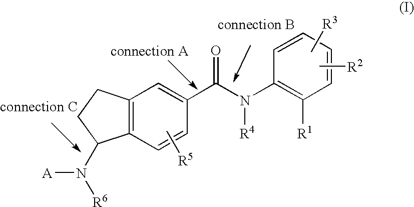

It should be apparent to those skilled in the art that the sequence of the synthetic steps is dependent on starting material availability and functional group compatibility and could vary from compound to compound. Protection and deprotection reactions could be involved in addition to the following reactions, as would be obvious to one skilled in the art. The groups A, and R¹ to R¹⁶ used below are as defined previously unless specified otherwise.

### Connection A

Connection A is the carbonylation of the optionally substituted indane portion of the molecule.

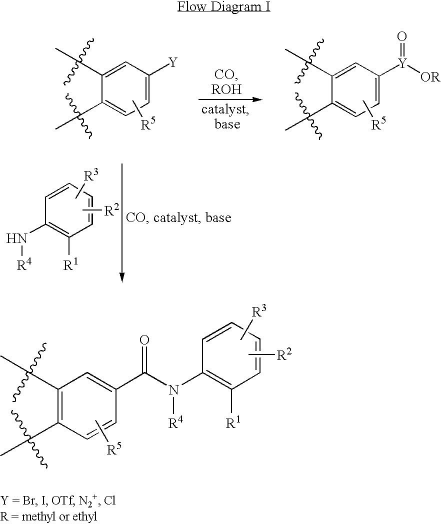

### Connection B

Connection B is the formation of amide between the optionally substituted propenoate and the optionally substituted aniline. It could be achieved by two routes outlined in Flow diagram II.

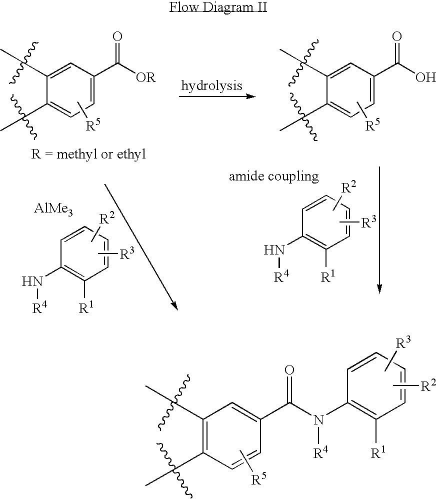

### Connection C

Connection C can be formed via the reductive amination of optionally substituted indanones or a reduction followed by further manipulation as illustrated in Flow Diagram III. The optionally substituted tryptamines are either commercially available or are prepared in similar manners as described in the literature procedures (for example, Tetrahedron Letters (2004), 45(15), 3123-3126; Journal of Medicinal Chemsitry, (1998), 41, 3831-3844; and Bioorganic & Medicinal Chemistry Letters (2003), 13, 1301-1305).

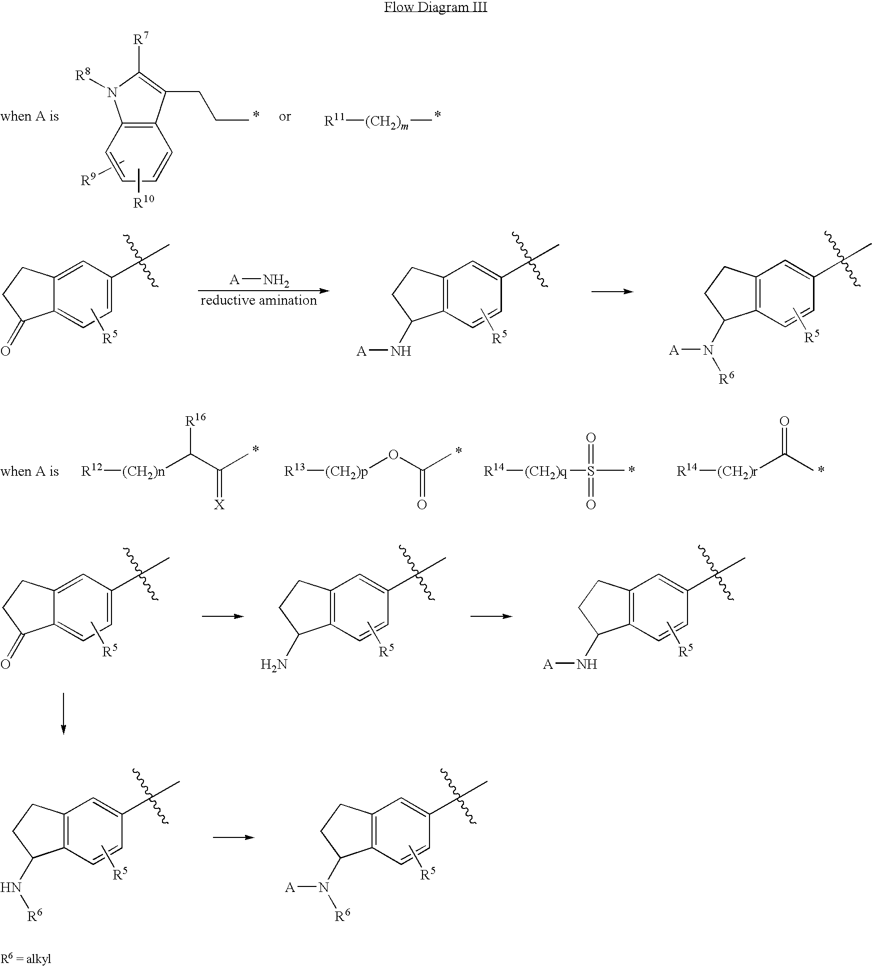

### Further Manipulations

If the following functional groups are present in the molecule, the transformations listed in Flow Diagram IV could be conducted.

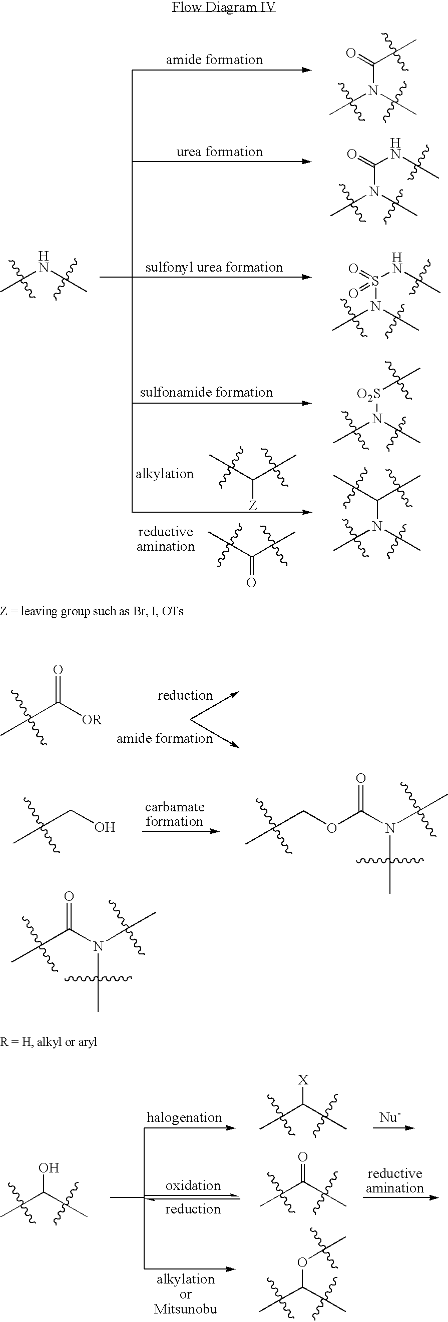

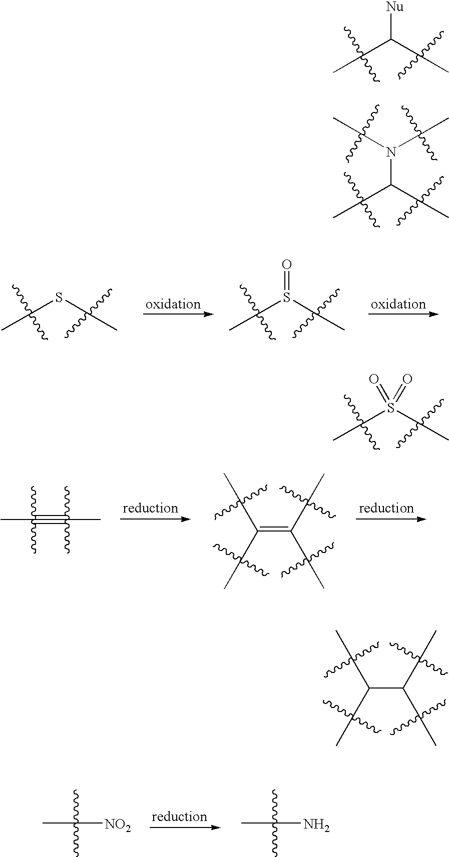

The compounds according to the invention exhibit useful pharmacological and pharmacokinetic properties. They are therefore suitable for use as medicaments for the treatment of disorders in humans and animals.

The compounds according to the invention are, because of their pharmacological properties, useful alone or in combination with other active components for treating or preventing hyper-proliferative disorders.

Another embodiment of the present invention relates to a method of using the compounds described above, including salts thereof and corresponding compositions thereof, to treat mammalian hyper-proliferative disorders. This method comprises administering to a patient an amount of a compound of this invention, or a pharmaceutically acceptable salt thereof, which is effective to treat the patient's hyper-proliferative disorder. A patient, for the purpose of this invention, is a mammal, including a human, in need of treatment for a particular hyper-proliferative disorder. Hyper-proliferative disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypothalamic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

In another embodiment, the present invention provides a medicament containing at least one compound according to the invention. In another embodiment, the present invention provides a medicament containing at least one compound according to the invention together with one or more pharmacologically safe excipient or carrier substances, and also their use for the abovementioned purposes.

The active compound can act systemically and/or locally. For this purpose it can be administered in a suitable manner, such as for example by oral, parenteral, pulmonary, nasal, sublingual, lingual, buccal, rectal, dermal, transdermal, ophthalmic or optic administration or in the form of an implant or stent. The active compound can be administered in forms suitable for these modes of administration.

Suitable forms of oral administration are those according to the prior art which function by releasing the active compound rapidly and/or in a modified or controlled manner and which contain the active compound in a crystalline and/or amorphous and/or dissolved form, such as for example tablets (which are uncoated or coated, for example with enteric coatings or coatings which dissolve after a delay in time or insoluble coatings which control the release of the active compound), tablets or films/wafers which disintegrate rapidly in the oral cavity or films/lyophilisates, capsules (e.g. hard or soft gelatin capsules), dragées, pellets, powders, emulsions, suspensions and solutions. An overview of application forms is given in Remington's Pharmaceutical Sciences, 18^{th} ed. 1990, Mack Publishing Group, Enolo.

Parenteral administration can be carried out by avoiding an absorption step (e.g. by intravenous, intraarterial, intracardial, intraspinal or intralumbar administration) or by including absorption (e.g. by intramuscular, subcutaneous, intracutaneous or intraperitoneal administration). Suitable parenteral administration forms are for example injection and infusion formulations in the form of solutions, suspensions, emulsions, lyophilisates and sterile powders. Such parenteral pharmaceutical compositions are described in Part 8, Chapter 84 of Remington's Pharmaceutical Sciences, 18^{th} ed. 1990, Mack Publishing Group, Enolo.

Suitable forms of administration for the other modes of administration are for example inhalation devices (such as for example powder inhalers, nebulizers), nasal drops, solutions and sprays; tablets or films/wafers for lingual, sublingual or buccal administration or capsules, suppositories, ear and eye preparations, vaginal capsules, aqueous suspensions (lotions or shaking mixtures), lipophilic suspensions, ointments, creams, transdermal therapeutic systems, milky lotions, pastes, foams, dusting powders, implants or stents.

The active compounds can be converted into the abovementioned forms of administration in a manner known to the skilled man and in accordance with the prior art using inert, non-toxic, pharmaceutically suitable auxiliaries. The latter include for example excipients (e.g. microcrystalline cellulose, lactose, mannitol, etc.), solvents (e.g. liquid polyethylene glycols), emulsifiers and dispersants or wetting agents (e.g. sodium dodecyl sulfate, polyoxysorbitan oleate etc.), binders (e.g. polyvinyl pyrrolidone), synthetic and/or natural polymers (e.g. albumin), stabilizers (e.g. antioxidants, such as, for example, ascorbic acid), dyes (e.g. inorganic pigments such as iron oxides) or taste- and/or odour-corrective agents.

The total amount of the active ingredient to be administered will generally range from about 0.01 mg/kg to about 200 mg/kg, and preferably from about 0.1 mg/kg to about 20 mg/kg body weight per day. A unit dosage may contain from about 0.5 mg to about 1500 mg of active ingredient, and can be administered one or more times per day. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

It may however be necessary to deviate from the abovementioned quantities, depending on the body weight, mode of administration, the individual patient response to the active compound, the type of preparation and the time or interval of administration.

If used as active compounds, the compounds according to the invention are preferably isolated in more or less pure form, that is more or less free from residues from the synthetic procedure. The degree of purity can be determined by methods known to the chemist or pharmacist (see Remington's Pharmaceutical Sciences, 18^{th} ed. 1990, Mack Publishing Group, Enolo). Preferably the compounds are greater than 99% pure (w/w), while purities of greater than 95%, 90% or 85% can be employed if necessary.

The percentages in the tests and examples which follows are, unless otherwise stated, by weight (w/w); parts are by weight. Solvent ratios, dilution ratios and concentrations reported for liquid/liquid solutions are each based on the volume.

### A. EXAMPLES

### Abbreviations and Acronyms

When the following abbreviations and symbols are used herein, they have the following meaning:

[α]_{D} optical rotation
AcOH acetic acid
Boc tert-butylcarboxy
CDI carbonyldiimidazole
DCM dichloromethane
DIBAL diisobutylaluminum hydride
DMAP 4-dimethylaminopyridine

### DMF N,N-dimethylformamide

DIPEA diisopropylethylamine
DMSO dimethylsulfoxide
dppf bis(diphenylphosphino)ferocene
dppp bis(diphenylphosphino)propane
EA elemental analysis
EDCI 1-(3-dimethylaminopropyl)-3 ethylcarbodiimide hydrochloride
ES electrospray
Et₃N triethylamine
Et₂O diethyl ether
EtOAc ethyl acetate
GC-MS Gas chromatography-mass spectrometry
h hour

### Hex Hexanes

HOBT 1-hydroxybenzotriazole hydrate
HPLC high performance liquid chromatography
iPrOH 2-propanol

### LC/MS Liquid Chromatography/Mass Spectrometry

Me methyl
MeOH methanol
min minutes
NaBH(OAc)₃ sodium triacetoxyborohydride

### NMR Nuclear Magnetic Resonance Spectroscopy

OTBDMS tert-butyl(dimethyl)silyloxy
OMe methoxy
Pd(OAc)₂ palladium (II) acetate
PyBOP Bromotripyrrolidinophosphonium hexafluorophosphate

### R_{f} TLC Retention Factor

RT retention time (HPLC)
rt room temperature
TBDMS tert-butyldimethylsilyl
TFA trifluoroacetic acid
THF tetrahydrofuran
TLC thin layer chromatography

### Experimental Procedures

### LC/MS Methods

HPLC-electrospray mass spectra (HPLC ES-MS) were obtained using a Hewlett-Packard 1100 HPLC equipped with a quaternary pump, a variable wavelength detector set at 254 nm, a YMC pro C-18 column (2×23 mm, 120 A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonirile with 0.018% TFA. Gradient elution from 10% B to 95% over 3.5 minutes at a flowrate of 1.0 μL/min was used with an initial hold of 0.5 minutes and a final hold at 95% B of 0.5 minutes. Total run time was 6.5 minutes.

### NMR Methods

Proton (¹H) nuclear magnetic resonance (NMR) spectra were measured with a Varian Mercury (300 MHz) or a Bruker Avance (500 MHz) spectrometer with either Me₄Si (δ0.00) or residual protonated solvent (CHCl₃ δ 7.26; MeOH δ 3.30; DMSO δ 2.49) as standard. The NMR data of the synthesized examples, some of which are not disclosed in the following detailed characterizations, are in agreements with their corresponding structural assignments.

### Optical Rotation

Optical rotations of the purified enantiomers were measured with a Perkin-Elmer 241 polarimeter under the sodium D line at room temperature. [α]_{D} was calculated and presented with the solvent and concentration used (g/100 mL).

Elemental analyses were conducted by Robertson Microlit Labs, Madison N.J. The results of elemental analyses, if conducted but not disclosed in the following detailed characterizations, are in agreements with their corresponding structural assignments.

### Preparation of Compound Example 2

### (±)-N-(2-aminophenyl)-1-{(2-hydroxyethyl)[2-(1H-indol-3-yl)ethyl]amino}indane-5-carboxamide

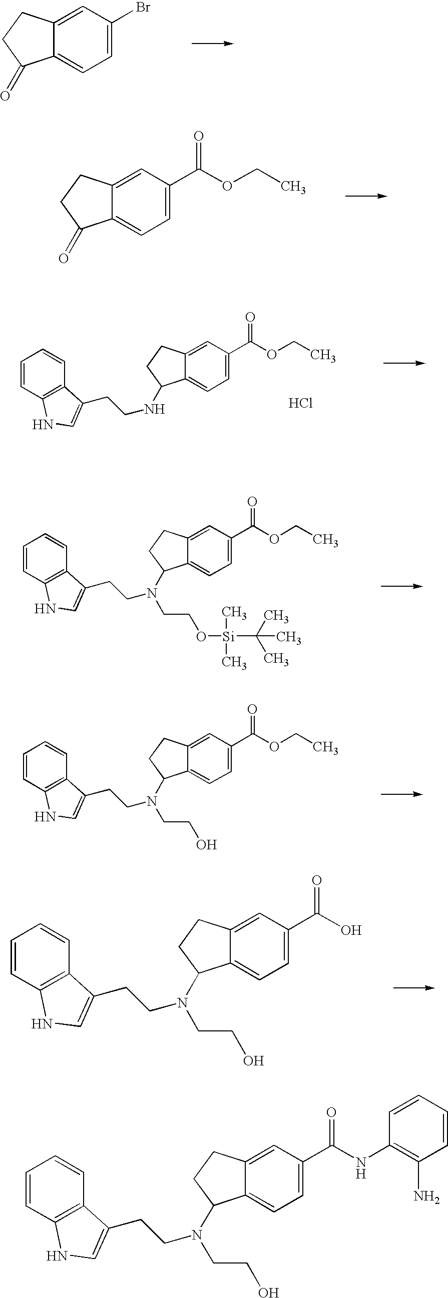

### Step 1: Preparation of Intermediate A, ethyl 1-oxoindane-5-carboxylate

To a solution of 5-bromo-1-indanone (200 mg, 0.95 mmol), 1,3-bis(diphenylphosphino)propane (98 mg, 0.24 mmol), EtOH (9 mL) and triethylamine (959 mg, 9.48 mmol) in DMF (9 mL) was added Pd(OAc)₂ (43 mg, 0.19 mmol). The resulting solution was stirred under one atmosphere of CO at 70° C. for 4 h. The reaction mixture was cooled to rt and diluted with water. The resulting mixture was extracted with EtOAc twice and the combined organic layer was washed with water and brine. The organic layer was dried over Na₂SO₄, filtered and concentrated under vacuum to obtain the crude product. It was then purified with 25 M biotage eluting with 15% EtOAc in hexane to obtain ethyl 1-oxoindane-5-carboxylate as a pale yellow solid (122 mg, 63%): ¹H-NMR (DMSO-d6) δ 8.12 (s, 1H), 7.92-7.95 (m, 1H), 7.73 (d, 1H), 4.32-4.37 (m, 2H), 3.17 (t, 2H), 2.68-2.72 (m, 2H), 1.35 (t, 3H).

### Step 2: Preparation of Intermediate B, (±)-ethyl 1-{[2-(1H-indol-3-yl)ethyl]amino}indane-5-carboxylate, hydrochloride salt

A mixture of tryptamine (114 mg, 0.71 mmol), intermediate A, ethyl 1-oxoindane-5-carboxylate (138 mg, 0.68 mmol) and titanium methoxide (233 mg, 1.36 mmol) in CH₂Cl₂ was stirred at rt overnight. NaBH(OAc)₃ (357 mg, 1.69 mmol) was added to the mixture and it was allowed to stir for another day. The reaction was quenched with 1N HCl (3 mL) and solid precipitated out of the solution, which was filtered and washed with CH₂Cl₂ to obtain ethyl 1-{[2-(1H-indol-3-yl)ethyl]amino}indane-5-carboxylate hydrochloride as a pale green solid (256 mg, 98%): LC/MS [M+H] 349.1, RT 2.44 min. ¹H-NMR (DMSO-d6) δ 10.95 (s, 1H), 9.51 (s, 1H), 9.36 (s, 1H), 7.84-7.89 (m, 3H), 7.57 (d, 1H), 7.35 (d, 1H), 7.24 (d, 1H), 7.6.97-7.09 (m, 2H), 4.88 (t, 1H), 4.28-4.33 (m, 2H), 2.91-3.23 (m, 6H), 2.47-2.54 (m, 1H), 2.22-2.27 (m, 1H), 1.32 (t, 3H).

### Step 3: Preparation of Intermediate C, (±)-ethyl 1-{(2-{[tert-butyl(dimethyl)silyl]oxy}ethyl) [2-(1H-indol-3-yl)ethyl]amino}indane-5-carboxylate

To a solution of intermediate B, ethyl 1-{[2-(1H-indol-3-yl)ethyl]amino}indane-5-carboxylate hydrochloride (500 mg, 1.30 mmol) in dichloroethane (15 mL) was added (tert-butyldimethylsilyloxy)acetaldehyde (249 mg, 1.43 mmol), AcOH (93 mg, 1.56 mmol) followed by NaBH(OAc)₃ (385 mg, 1.82 mmol). After 1 h at rt, saturated NaHCO₃ was added to quench the reaction and the resulting mixture was extracted with CH₂Cl₂ twice. The combined organic layer was washed with water, brine and concentrated to obtain the crude residue. It was then purified with 40 M Biotage eluting with 15% EtOAc in hexane to obtain ethyl 1-{(2-{[tert-butyl(dimethyl)silyl]oxy}ethyl)[2-(1H-indol-3-yl)ethyl]amino}indane-5-carboxylate as a colorless oil (572 mg, 87%): LC/MS [M+H] 507.3, RT 3.07 min. ¹H-NMR (DMSO-d6) δ 10.71 (s, 1H), 7.74-7.76 (m, 2H), 7.27-7.36 (m, 3H), 7.09 (d, 1H), 7.02 (t, 1H), 6.87 (t, 1H), 4.62 (t, 1H), 4.26-4.31 (m, 2H), 3.57-3.65 (m, 2H), 2.55-2.93 (m, 8H), 2.20-2.24 (m, 1H), 1.88-1.93 (m, 1H), 1.31 (t, 3H), 0.85 (s, 9H), 0.00 (s, 6H).

### Step 4: Preparation of Intermediate D, (±)-Ethyl 1-{(2-hydroxyethyl)[2-(1H-indol-3-yl)ethyl]amino}indane-5-carboxylate

To a solution of intermediate C, ethyl 1-{(2-{[tert-butyl(dimethyl)silyl]oxy}ethyl)[2-(1H-indol-3-yl)ethyl]amino}indane-5-carboxylate (570 mg, 1.12 mmol) in methanol (5 mL) was added 5% TFA in water (10 mL). The mixture was stirred at 40° C. for 2 h. The reaction was quenched with saturated NaHCO₃ and the mixture was extracted with EtOAc twice. The combined organic layer was washed with brine and concentrated to obtain the crude residue. It was purified with 25 M Biotage eluting with 50% EtOAc in hexane to obtain ethyl 1-{(2-hydroxyethyl)[2-(1H-indol-3-yl)ethyl]amino}indane-5-carboxylate as a colorless oil (392 mg, 89%): LC/MS [M+H] 393.2, RT 2.31 min. ¹H-NMR (DMSO-d6) δ 10.70 (s, 1H), 7.74-7.75 (m, 2H), 7.26-7.37 (m, 3H), 7.08 (d, 1H), 6.99 (t, 1H), 6.86 (t, 1H), 4.59 (t, 1H), 4.25-4.35 (m, 3H), 3.44-3.49 (m, 2H), 2.87-2.92 (m, 2H), 2.66-2.79 (m, 4H), 2.56 (t, 2H), 2.19-2.21 (m, 1H), 1.88-1.93 (m, 1H), 1.31 (t, 2H).

### Step 5: Preparation of Intermediate E, (±)-1-{(2-hydroxyethyl)[2-(1H-indol-3-yl)ethyl]amino}indane-5-carboxylic acid, hydrochloride salt

To a solution of intermediate D, ethyl 1-{(2-hydroxyethyl)[2-(1H-indol-3-yl)ethyl]amino}indane-5-carboxylate (371 mg, 0.95 mmol) in methanol (10 mL) was added aqueous KOH (529 mg in 2 mL of water), white solids precipritated out and THF (1 mL) was added. The mixture was stirred at rt overnight. 1N HCl was added to the reaction mixture to adjust the pH<1 and the mixture was extracted with ethyl acetate three times. The combined organic layer was dried over Na₂SO₄, filtered and concentrated under vacuo to give (±)-1-{(2-hydroxyethyl)[2-(1H-indol-3-yl)ethyl]amino}indane-5-carboxylic acid as an HCl salt (227 mg, 60%): LC/MS [M+H] 365.1, RT 1.83 min. ¹H-NMR (DMSO-d6) δ 10.89 and 10.98 (s, 1H), 10.22 (s, 1H), 7.80-8.02 (m, 3H), 7.20-7.41 (m, 2H), 6.93-7.21 (m, 3H), 5.26-5.55 (m, 2H), 3.83-4.25 (m, 4H), 3.48-3.82 (m, 2H), 2.87-3.23 (m, 5H), 2.69-2.86 (m, 1H).

### Step 6: Preparation of Compound Example 2, (±)-N-(2-aminophenyl)-1-{(2-hydroxyethyl) [2-(1H-indol-3-yl)ethyl]amino}indane-5-carboxamide

To a solution of 1-{(2-hydroxyethyl)[2-(1H-indol-3-yl)ethyl]amino}indane-5-carboxylic acid hydrochloride (121 mg, 0.30 mmol) in CH₂Cl₂ was added 1,2-phenylenediamine (75 mg, 0.69 mmol), EDCI (86 mg, 0.45 mmol), triethylamine (122 mg, 1.21 mmol) followed by HOBt (61 mg, 0.45 mmol). The mixture was stirred at rt overnight. The reaction was quenched with NaHCO₃ and extracted with CH₂Cl₂ twice. The combined organic layer was washed with brine and concentrated to give the crude product. It was purified with reverse phase preparative HPLC eluting with 10-50% CH₃CN in water in a flow rate of 25 mL/min. The corresponding fractions were combined, and free based with saturated NaHCO₃ and extracted with EtOAc. The organic layer was dried over Na₂SO₄, filtered and concentrated to give N-(2-aminophenyl)-1-{(2-hydroxyethyl) [2-(1H-indol-3-yl)ethyl]amino}indane-5-carboxamide as an oil (68 mg, 50%): LC/MS [M+H] 455.2, RT 1.98 min. ¹H-NMR (DMSO-d6) δ 10.71 (s, 1H), 9.56 (s, 1H), 7.76-7.78 (m, 2H), 7.27-7.38 (m, 3H), 7.10-7.14 (m, 2H), 6.88-7.02 (m, 3H), 6.74-6.76 (m, 1H), 6.53-6.60 (m, 1H), 4.86 (s, 2H), 4.61 (t, 1H), 4.34 (t, 1H), 3.45-3.51 (m, 2H), 2.70-2.94 (m, 6H), 2.57 (t, 2H), 2.21-2.25 (m, 1H), 1.91-1.98 (m, 1H). Compound example 1 was prepared similarly as described in steps 1, 2, and 6 under compound example 2.

### Preparation of Compound Example 3

### (±)-1-{acetyl[2-(1H-indol-3-yl)ethyl]amino}-N-(2-aminophenyl)indane-5-carboxamide

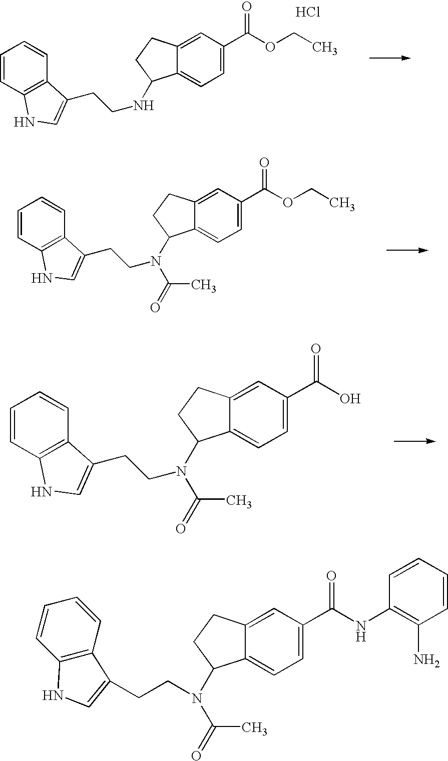

### Step 1: Preparation of Intermediate F, (±)-ethyl 1-{acetyl[2-(1H-indol-3-yl)ethyl]amino}indane-5-carboxylate

To a solution of intermediate B, ethyl 1-{[2-(1H-indol-3-yl)ethyl]amino}indane-5-carboxylate hydrochloride (200 mg, 0.52 mmol) in CH₂Cl₂ (5 mL) at 0° C. was added acetyl chloride (49 mg, 0.62 mmol) and Et₃N (79 mg, 0.78 mmol). The mixture was stirred at rt overnight. It was quenched with water and extracted with CH₂Cl₂ twice. The combined organic layer was concentrated and the crude product was purified with 25 S biotage eluting with 50% EtOAc in hexane to obtain ethyl 1-{acetyl[2-(1H-indol-3-yl)ethyl]amino}indane-5-carboxylate as a white solid (166 mg, 82%): LC/MS [M+H] 391.3, RT 3.24 min. ¹H-NMR (DMSO-d6) δ 10.79 and 10.68 (s, 1H), 7.76-7.85 (m, 2H), 6.76-7.36 (m, 6H), 5.86 and 5.55 (t, 1H), 4.26-4.32 (m, 2H), 2.73-3.43 (m, 6H), 2.37-2.46 (m, 1H), 2.15 and 2.22 (s, 3H), 1.98-2.03 (m, 1H), 1.31 (t, 3H).

### Step 2: Preparation of Intermediate G, (±)-1-{acetyl[2-(1H-indol-3-yl)ethyl]amino}indane-5-carboxylic acid

The reaction was performed similarly as described for steps 5 under compound example 2. LC/MS [M+H] 363.1, RT 2.69 min. ¹H-NMR (DMSO-d6) δ 12.82 (s, 1H), 10.69 and 10.81 (s, 1H), 7.70-7.88 (m, 2H), 6.75-7.39 (m, 7H), 5.55 and 5.87 (t, 1H), 3.17-3.50 (m, 2H), 2.68-3.13 (m, 4H), 2.30-2.55 (m, 1H), 2.22 and 2.15 (s, 3H), 1.94-2.15 (m, 1H).

### Step 3: Preparation of Compound Example 3

The reaction was performed similarly as described for step 6 under compound example 2. The product was isolated as a pair of rotomers: LC/MS [M+H] 453.2, RT 2.59 min. ¹H-NMR (DMSO-d6) δ 10.82 and 10.70 (s, 1H), 9.59-9.66 (d, 1H), 7.75-7.94 (m, 2H), 7.07-7.37 (m, 4H), 6.81-7.04 (m, 3H), 6.71-6.78 (dd, 1H), 6.60-6.61 (dd, 1H), 5.87-5.97 and 5.51-5.62 (m, 1H), 4.86 (s, 2H), 3.39-3.51 and 3.19-3.31 (m, 2H), 2.75-3.13 (m, 4H), 2.35-2.50 and 2.52-2.55 (m, 1H), 2.24 and 2.18 (s, 3H), and 1.98-2.15 (m, 2H).

### Preparation of Compound Example 4

### (±)-N-(2-aminophenyl)-1-{[(ethylamino)carbonyl][2-(1H-indol-3-yl)ethyl]amino}indane-5-carboxamide

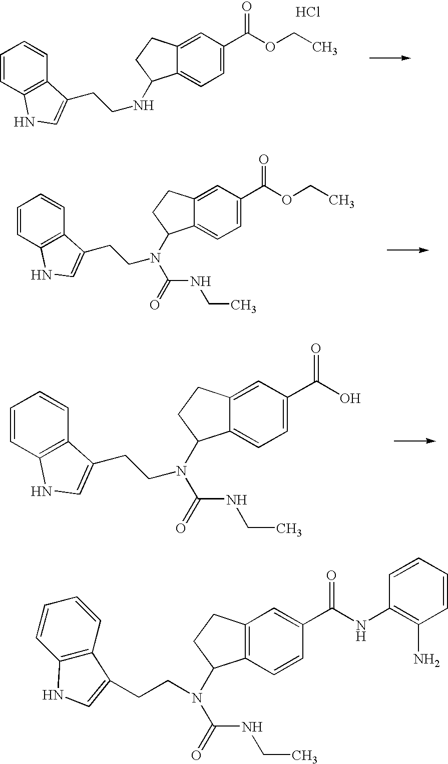

### Step 1: Preparation of Intermediate H, (±)-ethyl 1-{[(ethylamino)carbonyl][2-(1H-indol-3-yl)ethyl]amino}indane-5-carboxylate

To a solution of intermediate B, ethyl 1-{[2-(1H-indol-3-yl)ethyl]amino}indane-5-carboxylate hydrochloride (200 mg, 0.52 mmol) in CH₂Cl₂ (5 mL) at 0° C. was added ethyl isocyanate (41 mg, 0.57 mmol) and Et₃N (79 mg, 0.78 mmol). After 2 h at rt, the reaction was quenched with water and the mixture was extracted with CH₂Cl₂ twice. The combined organic layer was concentrated and the crude product was purified with 25 S Biotage eluting with 50% EtOAc in hexanes to obtain ethyl 1-{[(ethylamino)carbonyl][2-(1H-indol-3-yl)ethyl]amino}indane-5-carboxylate as a colorless oil (215 mg, 98%): LC/MS [M+H] 420.3, RT 3.32 min. ¹H-NMR (DMSO-d6) δ 10.70 (s, 1H), 7.78-7.81 (m, 2H), 7.22-7.25 (m, 2H), 7.12 (d, 1H), 6.95-7.00 (m, 2H), 6.79-6.83 (m, 1H), 6.40 (t, 1H), 5.68 (t, 1H), 4.25-4.31 (m, 2H), 3.10-3.21 (m, 3H), 2.95-3.02 (m, 2H), 2.75-2.85 (m, 3H), 2.33-2.37 (m, 1H), 1.92-1.95 (m, 1H), 1.30 (t, 3H), 1.17 (t, 3H).

### Step 2: Preparation of Intermediate I, (±)-1-{[(ethylamino)carbonyl][2-(1H-indol-3-yl)ethyl]amino}indane-5-carboxylic acid

The reaction was performed similarly as described for steps 5 under compound example 2. LC/MS [M+H] 392.3, RT 2.77 min. ¹H-NMR (DMSO-d6) δ 10.71 (s, 1H), 7.74-7.82 (m, 2H), 7.16-7.28 (m, 2H), 7.07-7.15 (d, 1H), 6.92-7.04 (m, 2H), 6.77-6.86 (t, 1H), 6.34-6.46 (m, 1H), 5.60-5.74 (t, 1H), 3.08-3.28 (m, 3H), 2.91-3.07 (m, 2H), 2.70-2.89 (m, 3H), 2.29-2.42 (m, 1H), 1.88-2.01 (m, 1H), 1.05 (t, 3H).

### Step 3: Preparation of Compound Example 4

The reaction was performed similarly as described for step 6 under compound example 2. LC/MS [M+H] 482.5, RT 2.69 min. ¹H-NMR (DMSO-d6) δ 10.72 (s, 1H), 9.60 (s, 1H), 7.78-7.88 (m, 2H), 7.09-7.28 (m, 4H), 6.82-7.04 (m, 4H), 6.71-6.77 (d, 1H), 6.56 (t, 1H), 6.43 (t, 1H), 5.71 (t, 1H), 4.88 (s, 2H), 3.11-3.31 (m, 3H), 2.94-3.07 (m, 2H), 2.77-2.91 (m, 3H), 2.30-2.44 (m, 1H), 1.88-2.03 (m, 1H), 1.08 (t, 3H).

### Preparation of Compound Example 5

### (±)-N-(2-aminophenyl)-1-[[2-(1H-indol-3-yl)ethyl](methylsulfonyl)amino]indane-5-carboxamide

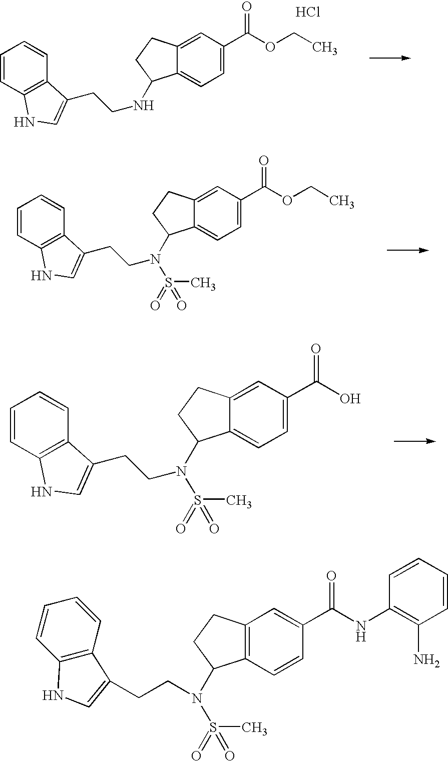

### Step 1: Preparation of Intermediate J, (±)-ethyl 1-[[2-(1H-indol-3-yl)ethyl](methylsulfonyl)amino]indane-5-carboxylate

To a solution of intermediate B, ethyl 1-{[2-(1H-indol-3-yl)ethyl]amino}indane-5-carboxylate hydrochloride (200 mg, 0.52 mmol) in CH₂Cl₂ (5 mL) at 0° C. was added methanesulfonyl chloride (71 mg, 0.62 mmol) and Et₃N (79 mg, 0.78 mmol). The mixture was stirred at rt overnight. The reaction was quenched with water and the mixture was extracted with CH₂Cl₂ twice. The combined organic layer was concentrated and the crude product was then purified with 25 S Biotage eluting with 40% EtOAc in hexane to obtain ethyl 1-[[2-(1H-indol-3-yl)ethyl](methylsulfonyl)amino]indane-5-carboxylate as a white solid (164 mg, 74%): LC/MS [M+H] 427.0, RT 3.42 min. ¹H-NMR (DMSO-d6) δ 10.73 (s, 1H), 7.85-7.87 (m, 2H), 7.51 (d, 1H), 7.24 (d, 1H), 6.94-6.98 (m, 2H), 6.85 (d, 1H), 6.76-6.78 (m, 1H), 5.43 (t, 1H), 4.28-4.33 (m, 2H), 3.32 (s, 3H), 2.87-3.18 (m, 6H), 2.61-2.68 (m, 1H), 2.04-2.09 (m, 1H), 1.32 (t, 3H).

### Step 2: Preparation of Intermediate K, (±)-1-[[2-(1H-indol-3-yl)ethyl](methylsulfonyl)amino]indane-5-carboxylic acid

The reaction was performed similarly as described for steps 5 under compound example 2. LC/MS [M+H] 398.8, RT 2.87 min. ¹H-NMR (DMSO-d6) δ 12.89 (s, 1H), 10.72 (s, 1H), 7.81-7.92 (m, 2H), 7.44-7.52 (d, 1H), 7.19-7.27 (d, 1H), 6.90-7.02 (m, 2H), 6.70-6.88 (m, 2H), 5.42 (t, 1H), 3.09-3.20 (m, 1H), 3.11 (s, 3H), 2.81-3.07 (m, 4H), 2.60-2.72 (m, 1H), 2.45-2.57 (m, 1H), 1.99-2.12 (m, 1H).

### Step 3: Preparation of Compound Example 5

The reaction was performed similarly as described for step 6 under compound example 2. LC/MS [M+H] 489.1, RT 2.79 min. ¹H-NMR (DMSO-d6) δ 10.74 (s, 1H), 9.67 (s, 1H), 7.86-7.95 (m, 2H), 7.46-7.52 (d, 1H), 7.21-7.26 (d, 1H), 7.10-7.17 (d, 1H), 6.88-7.02 (m, 4H), 6.72-6.86 (m, 2H), 6.57 (t, 1H), 5.44 (t, 1H), 4.88 (s, 2H), 3.12 (s, 3H), 2.84-3.13 (m, 5H), 2.63-2.77 (m, 1H), 2.47-2.59 (m, 1H), 2.03-2.15 (m, 1H).

### Preparation of Compound Example 6

### (±)-N-(2-aminophenyl)-1-{ethyl[2-(1H-indol-3-yl)ethyl]amino}indane-5-carboxamide

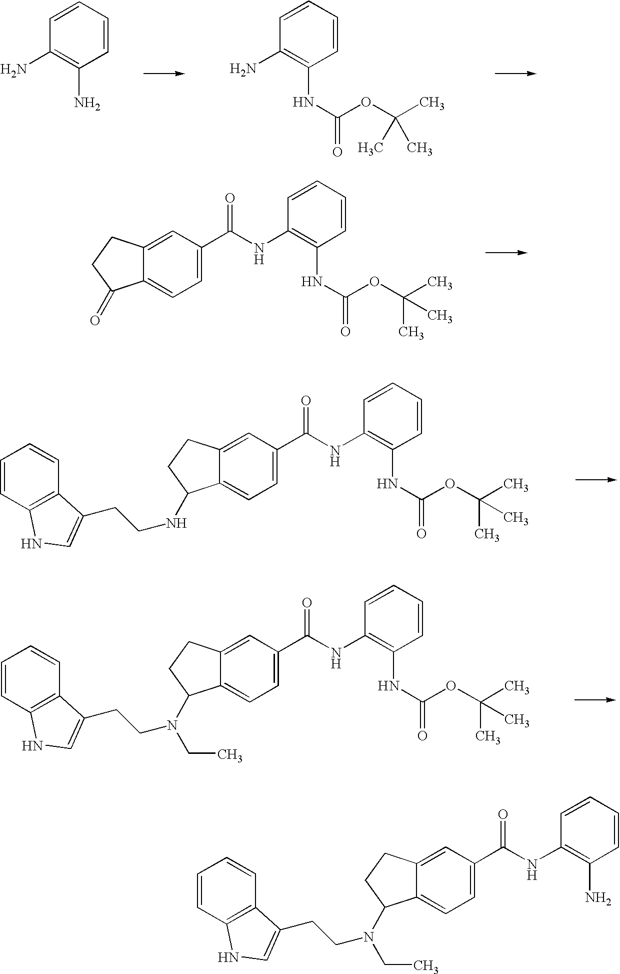

### Step 1: Preparation of Intermediate L, tert-butyl (2-aminophenyl)carbamate

To a cooled solution of benzene-1,2-diamine (30 g, 277.4 mmol) in THF (400 mL) was added di-tert-butyl dicarbonate (58.1 g, 266.3 mmol) slowly. The mixture was allowed to warm to rt and stirred overnight. The reaction was quenched with saturated NaHCO₃ and then the mixture was concentrated to remove most of the solvent. Water was added to the mixture and the organic layer was collected and then washed with brine, dried over Na₂SO₄, filtered and concentrated to give the crude material. The crude material was triturated with a mixture of ether in hexane (70%) twice to give tert-butyl (2-aminophenyl)carbamate as a white solid (43.8 g, 76%). LC/MS [M+H] 208.8, RT 1.51 min. ¹H-NMR (DMSO-d6) δ 8.258 (s, 1H), 7.156 (d, 1H), 6.784-6.826 (m, 1H), 6.638-6.661 (m, 1H), 6.474-6.515 (m, 1H), 4.803 (s, 2H), 1.452 (s, 9H).

### Step 2: Preparation of Intermediate M, tert-butyl (2-{[(1-oxo-2,3-dihydro-1H-inden-5-yl)carbonyl]amino}phenyl)carbamate

To a mixture of bromoindanone (1.8 g, 8.5 mmol), tert-butyl (2-aminophenyl)carbamate (3.6 g, 17.1 mmol), dppf (1.2 g, 2.1 mmol), DIPEA (4.4 g, 34.1 mmol) in DMF (20 mL) was added Pd(OAc)₂ (0.4 g, 1.7 mmol). The resultant solution was stirred under one atmosphere of carbon monoxide at 70° C. overnight. The reaction was quenched with water and then the mixture was passed through a pad of celite. The filtrate was then extracted with EtOAc three times. The combined organic layer was washed with brine, and then concentrated under vacuuo to give the crude residue. It was purified with 40 M biotage eluting with 20% EtOAc in hexane first and then 30% ethyl acetate in hexane to obtain a somewhat impure solid first. It was further purified by trituration with hexane to the pure product (1.16 g, 37%). LC/MS [M+Na] 389.1, RT 2.94 min. ¹H-NMR (DMSO-d6) δ 9.97 (s, 1H), 8.73 (s, 1H), 8.09 (s, 1H), 7.90-7.97 (d, 1H), 7.70-7.79 (d, 1H), 7.49-7.57 (m, 2H), 7.09-7.24 (m, 2H), 3.14-3.22 (m, 2H), 2.68-2.77 (m, 2H), 1.45 (s, 9H).

### Step 3, Preparation of Intermediate N, (±)-tert-butyl (2-{[(1-{[2-(1H-indol-3-yl)ethyl]amino}-2,3-dihydro-1H-inden-5-yl)carbonyl]amino}phenyl)carbamate

A mixture of tryptamine (230 mg, 1.4 mmol), tert-butyl (2-{[(1-oxo-2,3-dihydro-1H-inden-5-yl)carbonyl]amino}phenyl)carbamate (500 mg, 1.4 mmol) and Ti(OMe)₄ (470 mg, 2.7 mmol) in dichloromethane (250 mL) was stirred at rt over the weekend. NaBH(OAc)₃ (719.8 mg, 3.4 mmol) was added to the reaction mixture and it was left stirring at rt for 6 h. 1N HCl was added to quench the reaction and saturated NaHCO₃ was added to neutralize the mixture. It was then extracted with methylene chloride twice. The combined organic layer was washed with brine and concentrated under vacuo to give the crude residue. It was purified with 25 M eluting with 100% ethyl acetate to obtain tert-butyl(2-{[(1-{[2-(1H-indol-3-yl)ethyl]amino}-2,3-dihydro-1H-inden-5-yl)carbonyl]amino}phenyl)carbamate as a solid (549 mg, 79%). LC/MS [M+1] 511.2, RT 2.68 min. ¹H-NMR (DMSO-d6) δ 10.75 (s, 1H), 9.74 (s, 1H), 8.70 (s, 1H), 7.68-7.79 (m, 2H), 7.36-7.59 (m, 4H), 7.27-7.34 (d, 1H), 7.08-7.22 (m, 3H), 6.88-7.07 (m, 2H), 4.25 (t, 1H), 2.72-3.04 (m, 6H), 2.29-2.44 (m, 1H), 1.72-1.86 (m, 1H), 1.44 (s, 9H).

### Step 4, Preparation of Intermediate O, (±)-tert-butyl (2-{[(1-{ethyl[2-(1H-indol-3-yl)ethyl]amino}-2,3-dihydro-1H-inden-5-yl)carbonyl]amino}phenyl)carbamate

The reaction was performed similarly as described in step 3 under compound example 2: LC/MS [M+1] 539.2, RT 2.93 min. ¹H-NMR (DMSO-d6) δ 10.72 (s, 1H), 9.75 (s, 1H), 8.72 (s, 1H), 7.71-7.79 (m, 2H), 7.43-7.58 (m, 2H), 7.24-7.39 (m, 3H), 7.05-7.21 (m, 3H), 6.99 (t, 1H), 6.87 (t, 1H), 4.64 (t, 1H), 2.52-2.99 (m, 8H), 2.11-2.25 (m, 1H), 1.87-2.00 (m, 1H), 1.42 (s, 9H), 1.09 (t, 3H).

### Step 5, Preparation of Compound Example 6, (±)-N-(2-aminophenyl)-1-{ethyl[2-(1H-indol-3-yl)ethyl]amino}indane-5-carboxamide

A mixture of intermediate O, (±)-tert-butyl (2-{[(1-{ethyl[2-(1H-indol-3-yl)ethyl]amino}-2,3-dihydro-1H-inden-5-yl)carbonyl]amino}phenyl)carbamate (82 mg, 0.15 mmol) with TFA (1 mL) in CH₂Cl₂ (3 mL) was stirred at rt for 4 h. The reaction was concentrated under vacuo and the crude residue was purified with reverse phase preparative HPLC eluting with 10-50% CH3CN in water in a flow rate of 25 mL/min. The corresponding fractions were combined and free-based with saturated NaHCO₃. The organic layer was concentrated to give N-(2-aminophenyl)-1-{ethyl[2-(1H-indol-3-yl)ethyl]amino}indane-5-carboxamide as a solid (31 mg, 47%). LC/MS [M+1] 439.2, RT 2.09 min. ¹H-NMR (DMSO-d6) δ 10.71 (s, 1H), 9.57 (s, 1H), 7.73-7.82 (m, 2H), 7.24-7.38 (m, 3H), 7.06-7.17 (m, 2H), 6.83-7.05 (m, 3H), 6.72-6.78 (d, 1H), 6.58 (t, 1H), 4.87 (s, 2H), 4.63 (t, 1H), 2.49-3.01 (m, 8H), 2.12-2.24 (m, 1H), 1.87-2.01 (m, 1H), 1.09 (t, 3H).

Alternatively, 4N HCl (in 1,4-dioxane) in MeOH was used instead of TFA in CH₂Cl₂ to give similar results.

Compound examples 7-14 were prepared similarly as described in steps 1, 2, 3, and 5 under compound example 6.

### Preparation of Compound Example 15

### (±)-pyridin-3-ylmethyl (5-{[(2-aminophenyl)amino]carbonyl}-2,3-dihydro-1H-inden-1-yl)carbamate

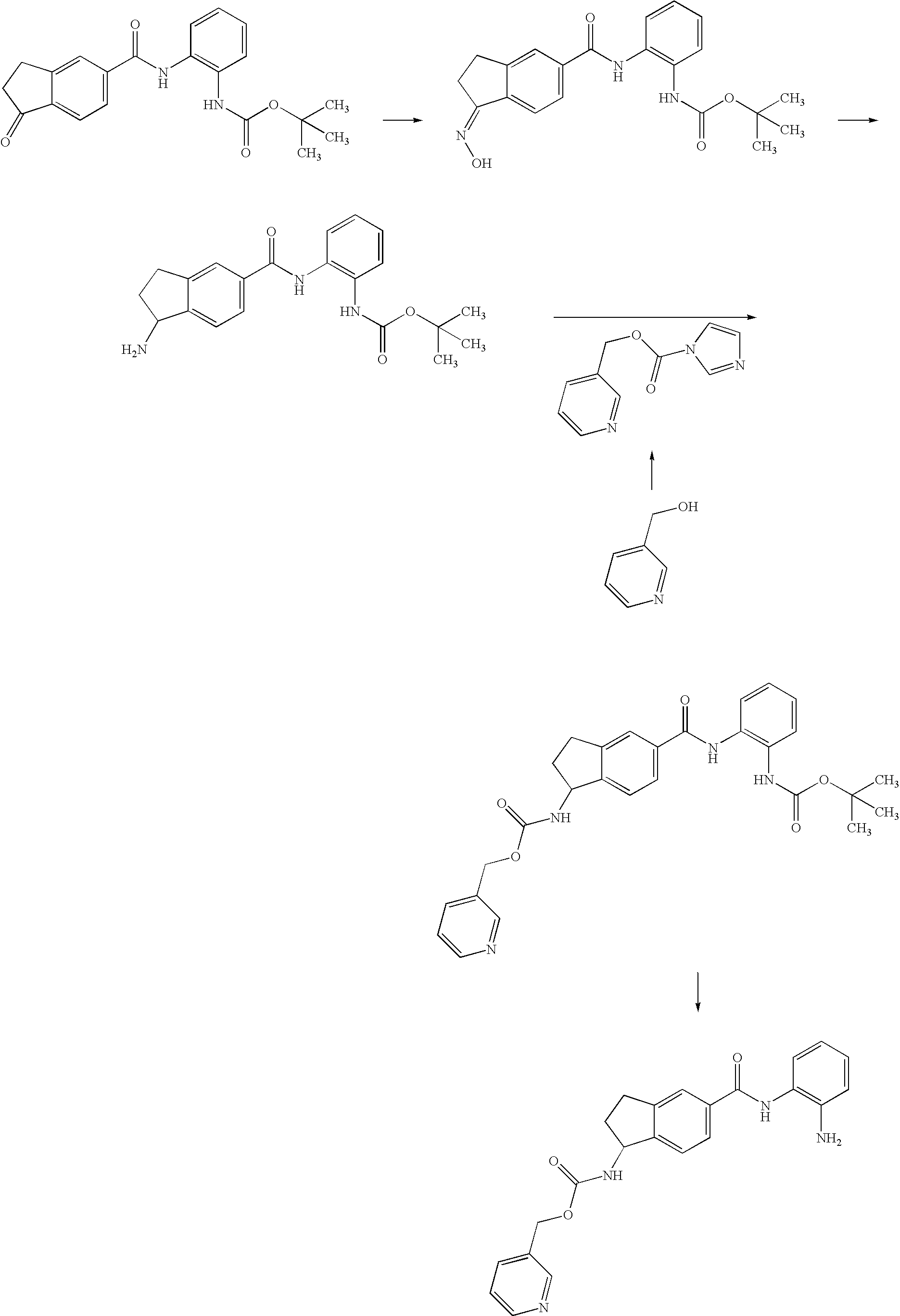

### Step 1: Preparation of Intermediate P, tert-butyl [2-({[(1Z)-1-(hydroxyimino)-2,3-dihydro-1H-inden-5-yl]carbonyl}amino)phenyl]carbamate

To a mixture of intermediate M, tert-butyl (2-{[(1-oxo-2,3-dihydro-1H-inden-5-yl)carbonyl]amino}phenyl)carbamate, (16.2 g, 44.2 mmol) and hydroxylamine hydrochloride (6.1 g, 88.4 mmol) in H₂O (14 μL)/EtOH (440 mL) was added NaOAc (7.3 g, 88.4 mmol). The resulting mixture was stirred overnight. In the morning, water (800 mL) was added to the thick suspension and the mixture was stirred for 15 min. The solid was collected by filtration, washed with H₂O two times, and dried under vacuum to give tert-butyl[2-({[(1Z)-1-(hydroxyimino)-2,3-dihydro-1H-inden-5-yl]carbonyl}amino)phenyl]carbamate as a white solid (16.8 g. 99% yield): LC/MS [M+H] 381.7, RT 3.02 min. ¹H-NMR (DMSO-d6) δ 11.11 (s, 1H), 9.84 (s, 1H), 8.71 (s, 1H), 7.89 (s, 1H), 7.80-7.84 (m, 1H), 7.65 (d, 1H), 7.47-7.55 (m, 2H), 7.10-7.20 (m, 2H), 3.03-3.09 (m, 2H), 2.82-2.87 (m, 2H), 1.43 (s, 9H).

### Step 2: Preparation of Intermediate Q, (±)-tert-butyl (2-{[(1-amino-2,3-dihydro-1H-inden-5-yl)carbonyl]amino}phenyl)carbamate

A 3-neck flask was flushed with nitrogen and palladium on activated carbon (Degussa type) (3.36 g) was added. While under a positive flow of N₂, 20 mL of MeOH was added to the flask. Intermediate P, tert-butyl [2-({[(1Z)-1-(hydroxyimino)-2,3-dihydro-1H-inden-5-yl]carbonyl}amino)phenyl]carbamate was dissolved in a mixture of MeOH (120 mL) and EtOAc (40 mL) and the resulting solution was added to the flask. The flask was then purged with hydrogen gas and the mixture was allowed to stir overnight. In the morning, the mixture was filtered through a pad of celite and the filtrate was concentrated. The crude material was purified by pad of silica gel eluting with 60% EtOAc/hexane to get rid of the non-polar impurities and then eluting with 5% (2N ammonia in MeOH)/methylene chloride to elute the product as a foamy solid (13.5 g, 83%, yield). LC/MS [M+H] 367.9, RT 2.18 min. ¹H-NMR (DMSO-d6) δ 9.17 (s, 1H), 8.71 (s, 1H), 7.71-7.80 (m, 2H), 7.53-7.57 (m, 1H), 7.44-7.48 (m, 2H), 7.10-7.19 (m, 2H), 4.22 (t, 1H), 2.86-2.92 (m, 1H), 2.70-2.81 (m, 1H), 2.34-2.43 (m, 1H), 2.08 (br s, 2H), 1.58-1.69 (m, 1H), 1.45 (s, 9H).

### Step 3: Preparation of Intermediate R, (±)-pyridin-3-ylmethyl {5-[({2-[(tert-butoxycarbonyl)amino]phenyl}amino)carbonyl]-2,3-dihydro-1H-inden-1-yl}carbamate

CDI (3.18 g, 19.6 mmol) was dissolved in THF (5 mL) and cooled to 0° C. 3-pyridylcarbinol (2.14 g, 19.6 mmol) was diluted with THF (5 mL) then added dropwise to the stirring solution of CDI. After 1 h, this mixture was added to a solution of intermediate Q, (±)-tert-butyl (2-{[(1-amino-2,3-dihydro-1H-inden-5-yl)carbonyl]amino}phenyl)carbamate (4.00 g, 10.9 mmol), Et₃N (1.5 mL, 10.9 mmol), and DBU (1.6 mL, 10.9 mmol) in THF (10 mL). The reaction was stirred overnight at rt. In the morning, the reaction mixture was diluted with EtOAc, washed with saturated sodium bicarbonate solution, and brine. The organic phase was collected, dried over Na₂SO₄, filtered, and concentrated under vacuum. The crude residue was purified by silica gel chromatography using a gradient of 60 to 85% EtOAc/Hexanes to give pyridin-3-ylmethyl {5-[({2-[(tert-butoxycarbonyl)amino]phenyl}amino)carbonyl]-2,3-dihydro-1H-inden-1-yl}carbamate (3.85 g, 70% yield). LC/MS [M+H] 503.0, RT 2.56 min. ¹H-NMR (DMSO-d6) δ 9.77 (s, 1H), 8.69 (br s, 1H), 8.59 (d, 1H), 8.52 (dd, 1H), 7.84 (d, 1H), 7.73-7.81 (m, 3H), 7.54 (dd, 1H), 7.40 (dd, 1H), 7.30 (d, 1H), 7.10-7.19 (m, 2H), 5.12 (s, 2H), 5.04-5.12 (m, 1H), 2.91-3.00 (m, 1H), 2.79-2.88 (m, 1H), 2.37-2.47 (m, 1H), 1.81-1.93 (m, 1H), 1.44 (s, 9H).

### Step 4: Preparation of Compound Example 15

The reaction was performed similarly as described in step 5 under compound example 6.

### Preparation of Compound Example 17

### (±)-N-(2-aminophenyl)-1-(ethyl{[(2-phenylethyl)amino]carbonyl}amino)indane-5-carboxamide

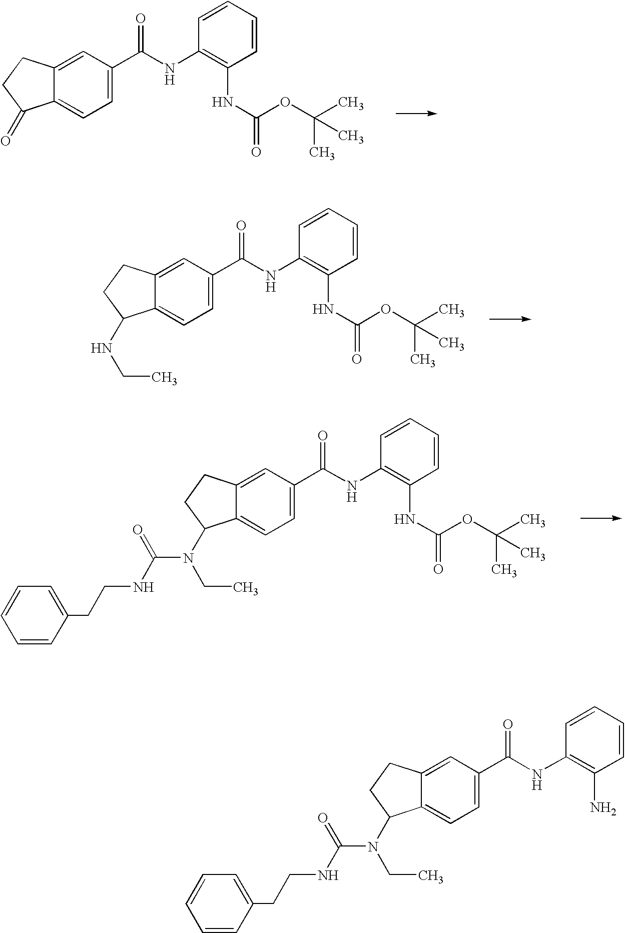

### Step 1, Preparation of Intermediate S, (±)-tert-butyl [2-({[1-(ethylamino)-2,3-dihydro-1H-inden-5-yl]carbonyl}amino)phenyl]carbamate

The reaction was performed similarly as described in step 2 under compound example 2. LC/MS [M+1] 396.1, RT 2.26 min. ¹H-NMR (DMSO-d6) δ 9.75 (s, 1H), 8.71 (s, 1H), 7.70-7.78 (m, 2H), 7.51-7.58 (m, 1H), 7.41-7.50 (m, 2H), 7.09-7.21 (m, 2H), 4.16 (t, 1H), 2.90-3.03 (m, 1H), 2.73-2.86 (m, 1H), 2.55-2.70 (m, 2H), 2.29-2.41 (m, 1H), 1.72-1.86 (m, 1H), 1.45 (s, 9H), 1.05 (t, 3H).

Step 2, Preparation of Intermediate T, (±)-tert-butyl [2-({[1-(ethyl{[(2-phenylethyl)amino]carbonyl}amino)-2,3-dihydro-1H-inden-5-yl]carbonyl}amino)phenyl]carbamate

The reaction was performed similarly as described in step 1 under compound example 4. LC/MS [M+Na] 565.2, RT 3.60 min. ¹H-NMR (DMSO-d6) δ 9.77 (s, 1H), 8.70 (s, 1H), 7.71-7.81 (m, 2H), 7.44-7.59 (dd, 2H), 7.07-7.34 (m, 8H), 6.37-6.45 (m, 1H), 5.71 (t, 1H), 3.27-3.36 (m, 2H), 2.71-3.13 (m, 6H), 2.26-2.38 (m, 1H), 1.88-2.03 (m, 1H), 1.45 (s, 9H), 0.95 (t, 3H).

### Step 3, Preparation of Compound Example 17

The reaction was performed similarly as described in step 5 under compound example 6. LC/MS [M+1] 443.2, RT 2.69 min. ¹H-NMR (DMSO-d6) δ 9.59 (s, 1H), 7.73-7.87 (m, 2H), 7.03-7.34 (m, 7H), 6.89-6.99 (m, 1H), 6.70-6.79 (d, 1H), 6.51-6.61 (m, 1H), 6.41 (t, 1H), 5.71 (t, 1H), 4.87 (s, 2H), 3.26-3.38 (m, 2H), 2.93-3.14 (m, 2H), 2.72-2.92 (m, 4H), 2.25-2.37 (m, 1H), 1.86-2.00 (m, 1H), 0.94 (t, 3H).

Compound examples 18 and 19 were prepared similarly as described under compound example 17.

### Preparation of Compound Example 20

### (±)-N-(2-aminophenyl)-1-[ethyl(phenylsulfonyl)amino]indane-5-carboxamide

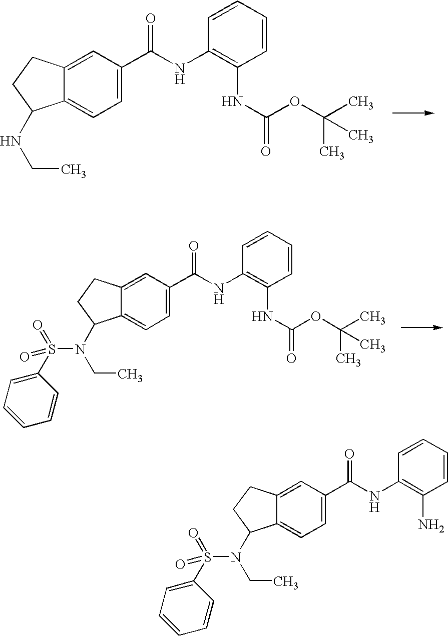

### Step 1: Preparation of Intermediate U, (±)-tert-butyl {2-[({1-[ethyl(phenylsulfonyl)amino]-2,3-dihydro-1H-inden-5-yl}carbonyl)amino]phenyl}carbamate

The reaction was performed similarly as described in step 1 under compound example 5. LC/MS [M+Na] 558.1, RT 3.78 min. ¹H-NMR (DMSO-d6) δ 9.76 (s, 1H), 8.71 (s, 1H), 7.89-7.96 (dd, 2H), 7.59-7.80 (m, 5H), 7.43-7.55 (m, 2H), 7.05-7.21 (m, 2H), 6.82-6.91 (dd, 1H), 5.50 (t, 1H), 2.88-3.02 (m, 3H), 2.75-2.88 (m, 1H), 2.13-2.27 (m, 1H), 1.69-1.83 (m, 1H), 1.43 (s, 9H), 0.99 (t, 3H).

### Step 2: Preparation of Compound Example 20

The reaction was performed similarly as described in step 5 under compound example 6. LC/MS [M+1] 436.2, RT 2.82 min. ¹H-NMR (DMSO-d6) δ 9.59 (s, 1H), 7.87-7.99 (d, 2H), 7.81 (s, 1H), 7.56-7.76 (m, 4H), 7.05-7.16 (d, 1H), 6.82-6.98 (m, 2H), 6.70-6.77 (dd, 1H), 6.55 (t, 1H), 5.49 (t, 1H), 4.86 (s, 2H), 2.89-3.04 (m, 3H), 2.73-2.87 (m, 1H), 2.14-2.25 (m, 1H), 1.67-1.84 (m, 1H), 0.99 (t, 3H). Compound examples 21-24, 28-30, 34, and 50-52 were prepared similarly as described for compound example 20.

### Preparation of Compound Example 25

### 1-[acetyl(ethyl)amino]-N-(2-aminophenyl)indane-5-carboxamide

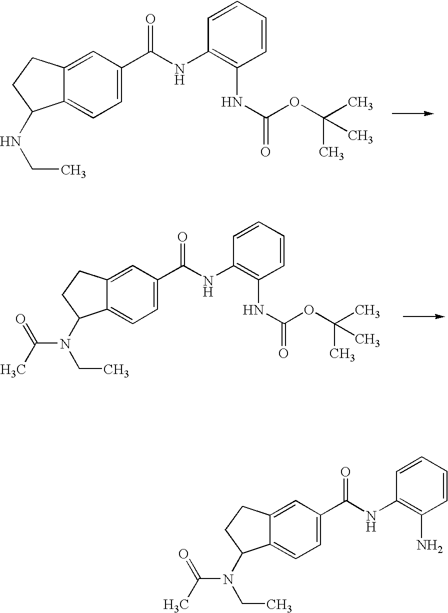

### Step 1: Preparation of Intermediate V, (±)-tert-butyl {2-[({1-[acetyl(ethyl)amino]-2,3-dihydro-1H-inden-5-yl}carbonyl)amino]phenyl}carbamate

The reaction was performed similarly as described in step 1 under compound example 3. The product was isolated as a pair of rotomers. LC/MS [M+1] 460.1, RT 3.08 min. ¹H-NMR (DMSO-d6) δ 9.76-9.84 (m, 1H), 8.64-8.79 (m, 1H), 7.69-7.88 (m, 2H), 7.43-7.58 (m, 2H), 7.07-7.34 (m, 3H), 5.49 and 5.87 (t, 1H), 3.20-3.34 and 2.78-2.96 (m, 2H), 2.97-3.17 (m, 2H), 2.11 and 2.16 (s, 3H), 1.95-2.09 and 2.24-2.39 (m, 2H), 1.42 (s, 9H), 0.97 and 1.05 (t, 3H).

### Step 2: Preparation of Compound Example 25

The reaction was performed similarly as described in step 5 under compound example 6. The product was isolated as a pair of rotomers. LC/MS [M+1] 338.2, RT 2.08 min. ¹H-NMR (DMSO-d6) δ 9.59 and 9.62 (s, 1H), 7.71-7.90 (m, 2H), 7.21-7.28 and 7.07-7.17 (dd, 2H), 6.89-6.98 (m, 1H), 6.71-6.79 (d, 1H), 6.51-6.62 (m, 1H), 5.87 and 5.48 (t, 1H), 4.88 (s, 2H), 3.20-3.34 and 2.78-2.96 (m, 2H), 2.97-3.17 (m, 2H), 2.11 and 2.16 (s, 3H), 1.95-2.09 and 2.24-2.39 (m, 2H), 0.97 and 1.05 (t, 3H).

Compound examples 26, 27, 31, and 32 were prepared similarly as described in compound example 25.

### Preparation of Compound Example 33

### (±)-4-fluorophenyl (5-{[(2-aminophenyl)amino]carbonyl}-2,3-dihydro-1H-inden-1-yl)ethylcarbamate

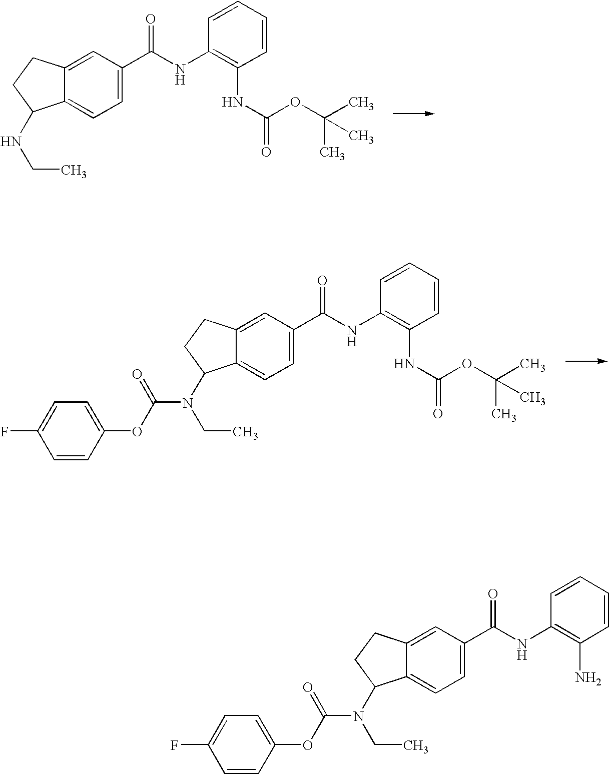

### Step 1: Preparation of Intermediate W, 4-fluorophenyl {5-[({2-[(tert-butoxycarbonyl)amino]phenyl}amino)carbonyl]-2,3-dihydro-1H-inden-1-yl}ethylcarbamate

A mixture of intermediate S (80.0 mg, 0.20 mmol), 4-fluorophenyl chlorofomate (42.4 mg, 0.24 mmol) and Et₃N (30.7 mg, 0.30 mmol) in DCM (3 mL) was stirred at rt overnight. In the morning, solvent was evaporated under vacuo and the crude product was purified with reverse phase preparative HPLC eluting with 15-95% CH3CN in water in a flow rate of 25 mL/min. The corresponding fractions were combined and free-based with saturated NaHCO₃, dried, and concentrated to give the desired product as a solid (66 mg, 61%). LC/MS [M+Na] 556.1, RT 3.93 min. 1H-NMR (DMSO-d6) δ 9.81 (s, 1H), 8.71 (s, 1H), 7.77-7.89 (m, 2H), 7.33-7.58 (m, 3H), 6.99-7.30 (m, 6H), 5.62 (t, 1H), 3.22-3.38 (m, 1H), 3.01-3.22 (m, 2H), 2.84-2.99 (m, 1H), 2.41-2.61 (m, 1H), 2.08-2.28 (m, 1H), 1.44 (s, 9H), 1.05-1.24 (m, 3H).

### Step 2, Preparation of Compound Example 33

The reaction was performed similarly as described in step 5 under compound example 6. LC/MS [M+1] 434.3, RT 2.96 min. ¹H-NMR (DMSO-d6) δ 9.63 (s, 1H), 7.78-7.91 (m, 2H), 7.28-7.46 (m, 1H), 7.00-7.26 (m, 5H), 6.90-6.99 (m, 1H), 6.71-6.79 (dd, 1H), 6.57 (t, 1H), 5.60 (t, 1H), 4.88 (s, 2H), 3.21-3.37 (m, 1H), 2.98-3.21 (m, 2H), 2.82-2.97 (m, 1H), 2.40-2.56 (m, 1H), 2.06-2.28 (m, 1H), 1.06-1.24 (m, 3H).

### Preparation of Compound Example 35

### (±)-N-(2-aminophenyl)-1-{[(2-methoxyphenyl)sulfonyl]amino}indane-5-carboxamide

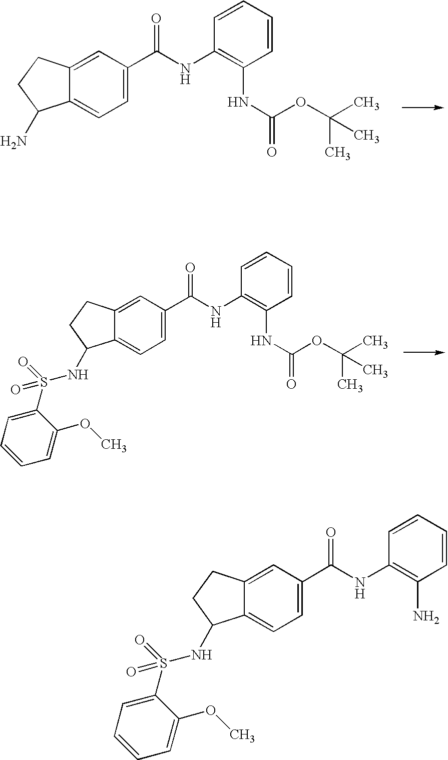

### Step 1: Preparation of Intermediate X, (±)-tert-butyl (2-{[(1-{[(2-methoxyphenyl)sulfonyl]amino}-2,3-dihydro-1H-inden-5-yl)carbonyl]amino}phenyl)carbamate

The reaction was performed similarly as described in step 1 under compound example 5. LC/MS [M+Na] 560.1, RT 3.51 min. ¹H-NMR (DMSO-d6) δ 9.77 (s, 1H), 8.71 (br s, 1H), 7.94 (d, 1H), 7.81 (dd, 1H), 7.71-7.75 (m, 2H), 7.60-7.65 (m, 1H), 7.54 (dd, 1H), 7.46 (dd, 1H), 4.73 (q, 1H), 3.89 (s, 3H), 2.84-2.93 (m, 1H), 2.66-2.77 (m, 1H), 2.03-2.12 (m, 1H), 1.74-1.85 (m, 1H), 1.44 (s, 9H).

### Step 2, Preparation of Compound Example 35

The reaction was performed similarly as described in step 5 under compound example 6. LC/MS [M+H] 438.2, RT 2.53 min. ¹H-NMR (DMSO-d6) δ 9.59 (s, 1H), 7.92 (d, 1H), 7.81 (dd, 1H), 7.75-7.78 (m, 2H), 7.60-7.65 (m, 1H), 7.24 (d, 1H), 7.18 (d, 1H), 7.06-7.15 (m, 2H), 6.91-6.96 (m, 1H), 6.75 (dd, 1H), 6.54-6.59 (m, 1H), 4.86 (s, 2H), 4.72 (q, 1H), 3.89 (s, 3H), 2.83-2.93 (m, 1H), 2.65-2.76 (m, 1H), 2.01-2.10 (m, 1H), 1.73-1.83 (m, 1H).

Compound examples 36-42 were prepared similarly as described for compound example 35.

### Preparation of Compound Example 43

### (±)-N-(2-aminophenyl)-1-({[(pyridin-4-ylmethyl)amino]carbonyl}amino)indane-5-carboxamide

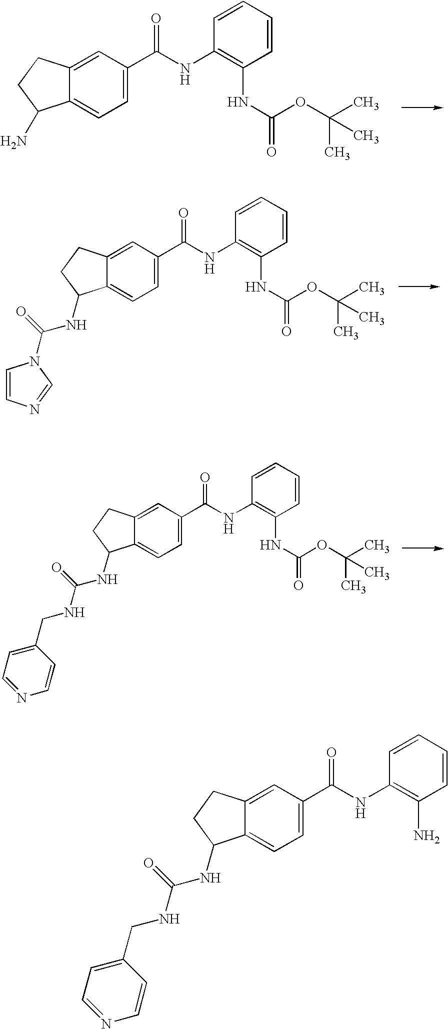

### Step 1, Preparation of Intermediate Y, (±)-tert-butyl {2-[({1-[(1H-imidazol-1-ylcarbonyl)amino]-2,3-dihydro-1H-inden-5-yl}carbonyl)amino]phenyl}carbamate

CDI (980 mg, 6.1 mmol) was suspended in THF (5 mL) and the mixture was cooled to 0° C. A solution of intermediate Q (2.0 g, 5.4 mmol) in THF (5 mL) was added dropwise to the stirring CDI. After 30 min, water and CH₂Cl₂ were added to the reaction. The organic layer was collected, dried over Na₂SO₄, and concentrated. The crude material was triturated in Et₂O to give (±)-tert-butyl {2-[({1-[(1H-imidazol-1-ylcarbonyl)amino]-2,3-dihydro-1H-inden-5-yl}carbonyl)amino]phenyl}carbamate as a white solid (2.3 g, 82% yield). LC/MS [M+H] 462.1, RT 2.58 min. ¹H-NMR (DMSO-d6) δ 9.81 (s, 1H), 8.87 (d, 1H), 7.72 (d, 1H), 8.29 (s, 1H), 7.78-7.85 (m, 2H), 7.73 (t, 1H), 7.56 (dd, 1H), 7.47 (dd, 1H), 7.45 (d, 1H), 7.11-7.20 (m, 2H), 7.03 (t, 1H), 5.45 (q, 1H), 3.03-3.12 (m, 1H), 2.89-2.99 (m, 1H), 2.52-2.61 (m, 1H), 2.02-2.12 (m, 1H), 2.45 (s, 9H).

### Step 2, Preparation of Intermediate Z, (±)-tert-butyl [2-({[1-({[(pyridin-4-ylmethyl)amino]carbonyl}amino)-2,3-dihydro-1H-inden-5-yl]carbonyl}amino)phenyl]carbamate

To a solution of intermediate Y, (±)-tert-butyl {2-[({1-[(1H-imidazol-1-ylcarbonyl)amino]-2,3-dihydro-1H-inden-5-yl}carbonyl)amino]phenyl}carbamate (88 mg, 0.19 mmol) in CH₂Cl₂ (2 mL) was added Et₃N (27 uL, 0.19 mmol) followed by 4-(aminomethyl)pyridine (21 mg, 0.19 mmol). The reaction mixture was stirred overnight. The reaction mixture was concentrated and the crude residue was purified by silica gel chromatography eluting with 5% MeOH/DCM to give (±)-tert-butyl [2-({[1-({[(pyridin-4-ylmethyl)amino]carbonyl}amino)-2,3-dihydro-1H-inden-5-yl]carbonyl}amino)phenyl]carbamate (58 mg, 61% yield). LC/MS [M+H] 502.1, RT 2.34 min. ¹H-NMR (DMSO-d6) δ 9.78 (s, 1H), 8.71 (br s, 1H), 8.47-8.50 (m, 2H), 7.75-7.79 (m, 2H), 7.55 (dd, 1H), 7.47 (dd, 1H), 7.32 (d, 1H), 7.24-7.27 (m, 2H), 7.11-7.20 (m, 2H), 6.57 (d, 1H), 6.49 (t, 1H), 5.17 (q, 1H), 4.29 (d, 2H), 2.91-2.99 (m, 1H), 2.79-2.89 (m, 1H), 2.41-2.49 (m, 1H), 1.74-1.84 (m, 1H), 1.45 (s, 9H).

### Step 3, Preparation of Compound Example 43

The reaction was performed similarly as described in step 5 under compound example 6. LC/MS [M+H] 402.2, RT 1.10 min. ¹H-NMR (DMSO-d6) δ 9.59 (s, 1H), 8.46-8.51 (m, 2H), 7.77-7.83 (m, 2H), 7.29 (d, 1H), 7.24-7.27 (m, 2H), 7.14 (d, 1H), 6.92-6.97 (m, 1H), 6.76 (d, 1H), 6.53-6.60 (m, 2H), 6.45 (t, 1H), 5.17 (q, 1H), 4.87 (s, 2H), 4.29 (d, 1H), 2.91-2.30 (m, 1H), 2.78-2.88 (m, 1H), 2.41-2.49 (m, 1H), 2.73-2.83 (m, 1H).

Compound examples 16, 44-47, 60-71, 79-112, 115-121 were prepared similarly as described for compound example 43.

### Preparation of Compound Example 48

### (±)-phenyl (5-{[(2-aminophenyl)amino]carbonyl}-2,3-dihydro-1H-inden-1-yl)carbamate

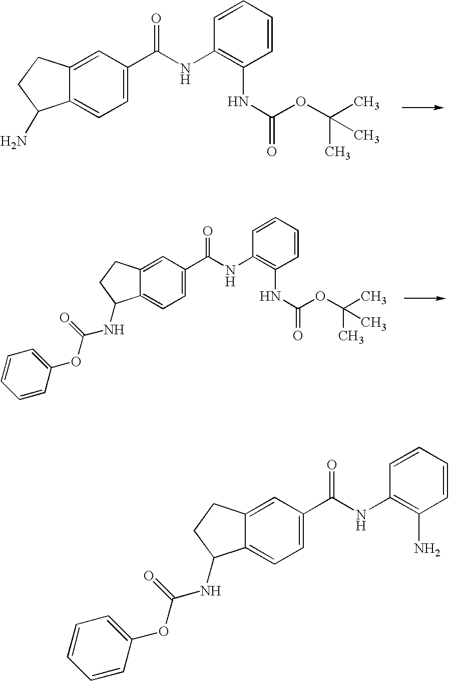

### Step 1: Preparation of Intermediate AA, phenyl {5-[({2-[(tert-butoxycarbonyl)amino]phenyl}amino)carbonyl]-2,3-dihydro-1H-inden-1-yl}carbamate

The reaction was performed similarly as described in step 1 under compound example 33. LC/MS [M+Na] 510.1, RT 3.59 min. ¹H-NMR (DMSO-d6) δ 9.80 (s, 1H), 8.71 (br s, 1H), 8.29 (d, 1H), 7.70-7.84 (m, 2H), 7.55 (dd, 1H), 7.48 (dd, 1H), 7.36-7.45 (m, 3H), 7.11-7.23 (m, 5H), 5.13 (q, 1H), 2.97-3.06 (m, 1H), 2.83-2.93 (m, 1H), 2.46-2.55 (m, 1H), 1.92-2.02 (m, 1H), 1.45 (s, 9H).

### Step 2, Preparation of Compound Example 48

The reaction was performed similarly as described in step 5 under compound example 6. LC/MS [M+H] 388.1, RT 2.57 min. ¹H-NMR (DMSO-d6) δ 9.61 (s, 1H), 8.28 (d, 1H), 7.79-7.87 (m, 2H), 7.35-7.42 (m, 3H), 7.12-7.24 (m, 4H), 6.92-6.97 (m, 1H), 6.74-6.78 (m, 1H), 6.55-6.60 (m, 1H), 5.12 (q, 1H), 4.88 (s, 2H), 3.38-3.47 (m, 1H), 2.96-3.06 (m, 1H), 2.83-2.91 (m, 1H), 1.91-2.01 (m, 1H).

Compound example 49 was prepared similarly as described for compound example 48.

### Preparation of Compound Example 53

### (±)-1-(acetylamino)-N-(2-aminophenyl)indane-5-carboxamide

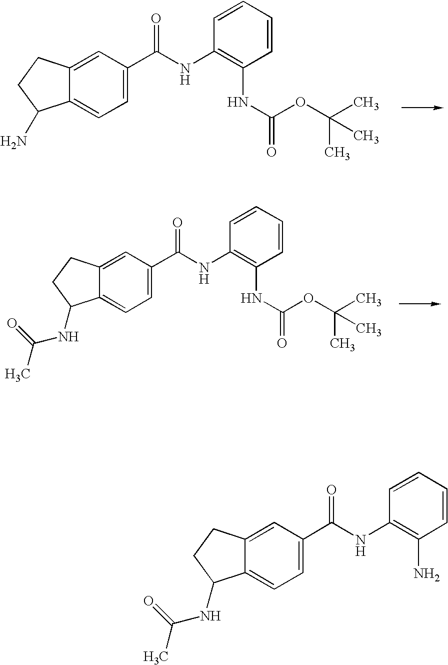

### Step 1: Preparation of Intermediate AB, (±)-tert-butyl [2-({[1-(acetylamino)-2,3-dihydro-1H-inden-5-yl]carbonyl}amino)phenyl]carbamate

The reaction was performed similarly as described in step 1 under compound example 3. LC/MS [M+Na] 432.1, RT 2.84 min. ¹H-NMR (DMSO-d6) δ 9.78 (s, 1H), 8.71 (br s, 1H), 8.28 (d, 1H), 7.74-7.79 (m, 2H), 7.53-7.56 (dd, 1H), 7.45-7.49 (dd, 1H), 7.30 (d, 1H), 7.11-7.19 (m, 2H), 5.30 (q, 1H), 2.94-3.02 (m, 1H), 2.81-2.89 (m, 1H), 2.38-2.46 (m, 1H), 1.89 (s, 3H), 1.77-1.87, 1H), 1.45 (s, 9H).

### Step 2, Preparation of Compound Example 53

The reaction was performed similarly as described in step 5 under compound example 6. LC/MS [M+H] 310.1, RT 1.40 min. ¹H-NMR (DMSO-d6) δ 9.59 (s, 1H), 8.27 (d, 1H), 7.82 (s, 1H), 7.78 (d, 1H), 7.27 (d, 1H), 7.14 (d, 1H), 6.92-6.97 (ddd, 1H), 6.74-6.77 (dd, 1H), 6.55-6.60 (ddd, 1H), 5.29 (q, 1H), 4.87 (d, 2H), 2.93-3.02 (m, 1H), 2.81-2.89 (m, 1H), 2.37-2.46 (m, 1H), 1.89 (s, 1H), 1.76-1.86 (m, 1H).

Compound examples 54-56 were prepared similarly as described for compound example 53.

### Preparation of Compound Example 57

### (±)-3-phenylpropyl (5-{[(2-aminophenyl)amino]carbonyl}-2,3-dihydro-1H-inden-1-yl)carbamate

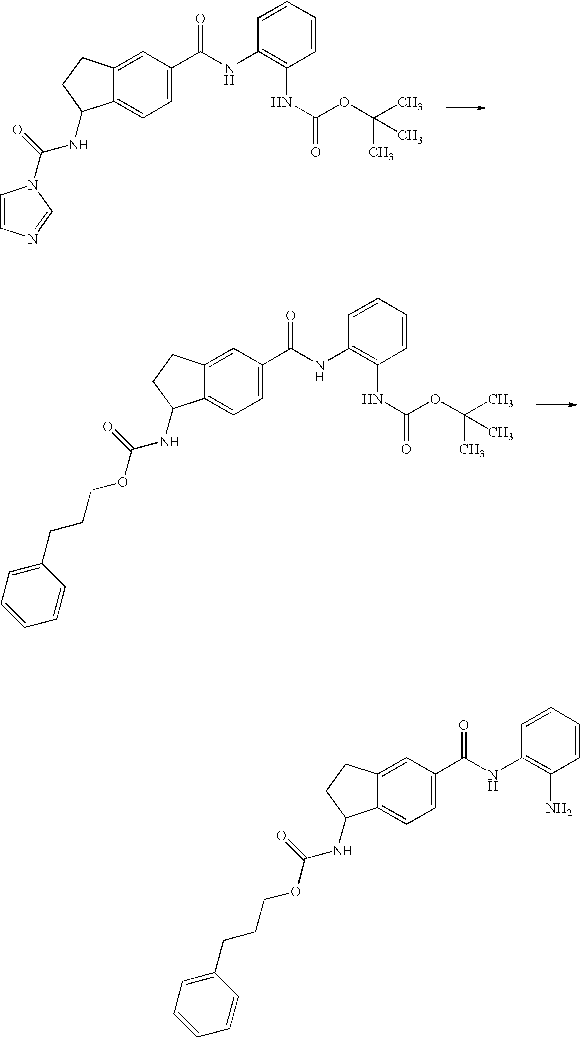

### Step 1: Preparation of Intermediate AC, 3-phenylpropyl {5-[({2-[(tert-butoxycarbonyl)amino]phenyl}amino)carbonyl]-2,3-dihydro-1H-inden-1-yl}carbamate

3-phenyl-1-propanol (33.0 mg, 0.24 mmol) was added to a stirring suspension of NaH (60% suspension in mineral oil, 11.0 mg, 0.28 mmol) in THF (1 mL). After 30 min. stirring, intermediate Y, (±)-tert-butyl {2-[({1-[(1H-imidazol-1-ylcarbonyl)amino]-2,3-dihydro-1H-inden-5-yl}carbonyl)amino]phenyl}carbamate (86 mg, (0.19 mmol) was added as a solution in THF (1 mL). The reaction was stirred for 2 h at which time a drop of MeOH was added to quench the reaction. The resulting mixture was concentrated and the residue was purified by a silica gel column eluting with a gradient of EtOAc/Hex (0% to 45% give 3-phenylpropyl {5-[({2-[(tert-butoxycarbonyl)amino]phenyl}amino)carbonyl]-2,3-dihydro-1H-inden-1-yl}carbamate (9.3 mg, 37% yield). LC/MS [M+Na] 552.2, RT 3.87 min.

### Step 2: Preparation of Compound Example 57

The reaction was performed similarly as described in step 5 under compound example 6. LC/MS [M+H] 430.2, RT 2.97 min. ¹H-NMR (DMSO-d6) δ 9.59 (s, 1H), 7.76-7.83 (m, 3H), 7.65 (d, 1H), 7.24-7.31 (m, 3H), 7.11-7.22 (m, 4H), 6.92-6.96 (ddd, 1H), 6.74-6.77 (dd, 1H), 6.55-6.60 (ddd, 1H), 5.06 (q, 1H), 4.87 (s, 2H), 4.00 (t, 2H), 2.92-3.01 (m, 1H), 2.78-2.87 (m, 1H), 2.66 (t, 2H), 2.38-2.47 (m, 1H), 1.77-1.93 (m, 3H).

Compound examples 58-59, 74-78 were prepared similarly as described for compound example 57.

### Preparation of Compound Examples 72, (−)-pyridin-3-ylmethyl (5-{[(2-aminophenyl)amino]carbonyl}-2,3-dihydro-1H-inden-1-yl)carbamate and 73, (+)-pyridin-3-ylmethyl (5-{[(2-aminophenyl)amino]carbonyl}-2,3-dihydro-1H-inden-1-yl)carbamate

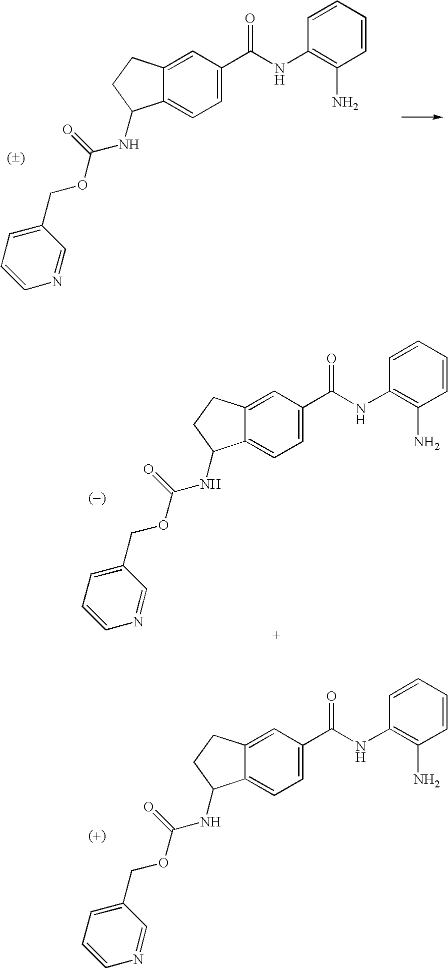

Racemic compound example 15, (±)-pyridin-3-ylmethyl (5-{[(2-aminophenyl)amino]carbonyl}-2,3-dihydro-1H-inden-1-yl)carbamate (3.00 g) was separated with Chiracel OD-H 20×250 mm using 50% (1:1 MeOH/EtOH) in heptane with 0.2% Et₃N (flow rate=15 mL/min) to obtain the (−)-isomer (RT=11.20 min, 1.20 g): [α]_{D} (MeOH)=−65.0 (c, 1.1) and the (+)-isomer (RT=15.00 min, 1.20 g): [α]_{D} (MeOH)=71.6 (c, 1.2). The overall recovery yield was 80%.

### Preparation of Compound Example 113

### (±)-N-(2-aminophenyl)-1-[(anilinocarbonothioyl)amino]indane-5-carboxamide

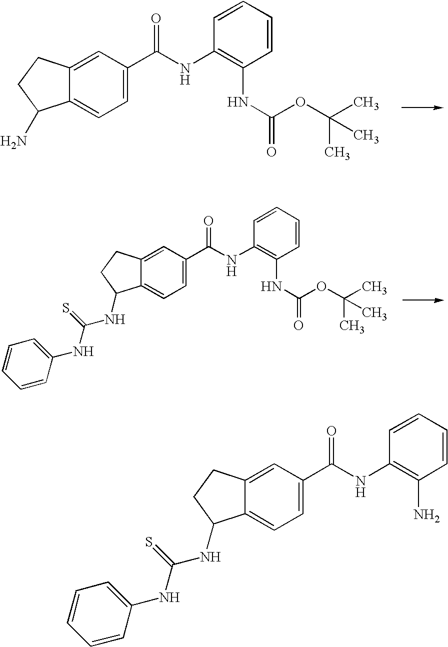

### Step 1: Preparation of Intermediate AD, (±)-tert-butyl {2-[({1-[(anilinocarbonothioyl)amino]-2,3-dihydro-1H-inden-5-yl}carbonyl)amino]phenyl}carbamate

To a solution of intermediate Q (150 mg, 0.14 mmol) in THF (4 mL) at 0° C. was added phenylthioisocyanate (60 mg, 0.45 mmol). After 30 min., the reaction mixture was concentrated and purified by a silica gel column eluting with a gradient of EtOAc/Hex (0 to 50%) to give tert-butyl {2-[({1-[(anilinocarbonothioyl)amino]-2,3-dihydro-1H-inden-5-yl}carbonyl)amino]phenyl}carbamate (125 mg, 61% yield). LC/MS [M+H] 502.9, RT 3.57 min. ¹H-NMR (DMSO-d6) δ 9.79 (s, 1H), 9.55 (s, 1H), 8.71 (br s, 1H), 8.15 (d, 1H), 7.77-7.82 (m, 2H), 7.53-7.57 (dd, 1H), 7.44-7.51 (m, 4H), 7.27-7.33 (m, 2H), 7.12-7.19 (m, 2H), 7.06-7.12 (m, 1H), 5.95-6.03 (m, 1H), 4.88 (s, 2H), 2.95-3.04 (m, 1H), 2.85-2.94 (m, 1H), 2.53-2.62 (m, 1H), 1.90-2.00 (m, 1H), 1.45 (s, 9H).

### Step 2: Preparation of Compound Example 113

The reaction was performed similarly as described in step 5 under compound example 6. LC/MS [M+H] 403.0, RT 2.58 min. ¹H-NMR (DMSO-d6) δ 9.61 (s, 1H), 9.54 (s, 1H), 8.15 (d, 1H), 7.79-7.86 (m, 2H), 7.43-7.50 (m, 3H), 7.27-7.33 (m, 2H), 7.14 (d, 1H), 7.08 (t, 1H), 6.92-6.97 (ddd, 1H), 6.74-6.77 (dd, 1H), 6.55-6.60 (ddd, 1H), 5.93-6.00 (m, 1H), 4.88 (s, 2H), 2.95-3.04 (m, 1H), 2.85-2.93 (m, 1H), 2.53-2.61 (m, 1H), 1.89-1.99 (m, 1H).

### Preparation of Compound Example 114

### (±)-N-(2-aminophenyl)-1-(1,3-benzothiazol-2-ylamino)indane-5-carboxamide

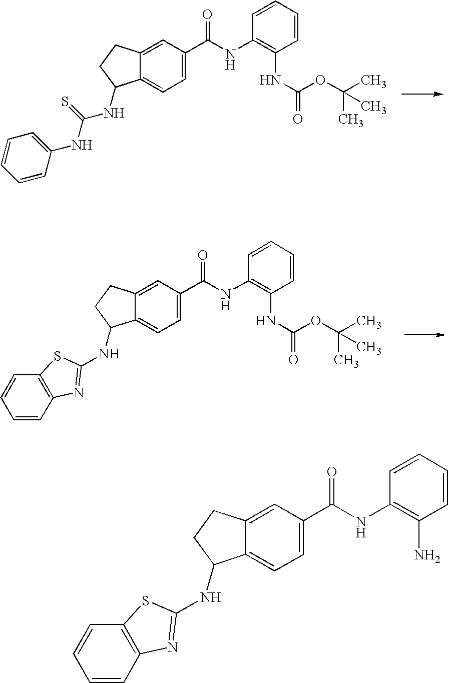

### Step 1: Preparation of Intermediate AE, (±)-tert-butyl [2-({[1-(1,3-benzothiazol-2-ylamino)-2,3-dihydro-1H-inden-5-yl]carbonyl}amino)phenyl]carbamate

Sulfuryl chloride (44 mg, 0.32 mmol) was added dropwise to a solution of intermediate AD (125 mg, 0.25 mmol) in CH₂Cl₂ (3 mL) at 0° C. After 5 min, the reaction was quenched with water and the mixture was extracted with DCM. The organic layer was collected, freebased with ammonia (2N in MeOH), and concentrated. The crude residue was purified by silica gel chromatography using a gradient of EtOAc/Hexanes (0% to 50% to give tert-butyl [2-({[1-(1,3-benzothiazol-2-ylamino)-2,3-dihydro-1H-inden-5-yl]carbonyl}amino)phenyl]carbamate (78 mg, 63% yield). LC/MS [M+H] 501.0, RT 3.24 min. ¹H-NMR (DMSO-d6) δ 9.80 (s, 1H), 8.70 (br s, 1H), 8.45 (d, 1H), 7.83 (s, 1H), 7.75-7.79 (m, 1H), 7.66-7.69 (m, 1H), 7.53-7.57 (m, 1H), 7.40-7.50 (m, 3H), 7.20-7.25 (m, 1H), 7.11-7.19 (m, 2H), 7.01-7.05 (m, 1H), 5.54 (q, 1H), 4.87 (s, 2H), 3.01-3.09 (m, 1H), 2.89-2.97 (m, 1H), 2.59-2.68 (m, 1H), 1.93-2.02 (m, 1H), 1.45 (s, 9H).

### Step 2, Preparation of Compound Example 114

The reaction was performed similarly as described in step 5 under example 6. LC/MS [M+H] 401.0, RT 2.37 min. ¹H-NMR (DMSO-d6) δ 9.61 (s, 1H), 8.45 (d, 1H), 7.87 (s, 1H), 7.78 (d, 1H), 7.66-7.69 (dd, 1H), 7.34-7.44 (m, 2H), 7.20-7.25 (ddd, 1H), 7.14 (d, 1H), 7.00-7.05 (ddd, 1H), 6.92-6.96 (ddd, 1H), 6.74-6.77 (dd, 1H), 6.55-6.60 (ddd, 1H), 5.53 (q, 1H), 3.00-3.09 (m, 1H), 2.88-2.96 (m, 1H), 2.59-2.66 (m, 1H), 1.92-2.01 (m, 1H).

### Preparation of Compound Example 122

### (±)-N-(2-aminophenyl)-1-[(3-pyridin-3-ylpropanoyl)amino]indane-5-carboxamide

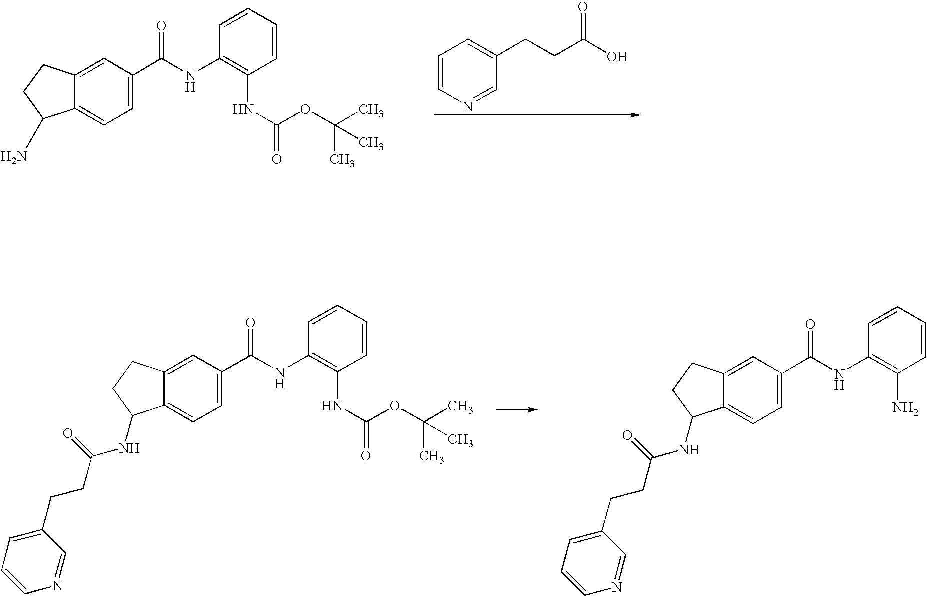

### Step 1: Preparation of Intermediate AF, tert-butyl {2-[({1-[(3-pyridin-3-ylpropanoyl)amino]-2,3-dihydro-1H-inden-5-yl}carbonyl)amino]phenyl}carbamate

Intermediate Q (100 mg, 0.27 mmol), 3-pyridylpropionic acid (49 mg, 0.33 mmol), EDCI (78 mg, 0.41 mmol), HOBT (55 mg, 0.41 mmol), and Et₃N (76 ul, 0.54 mmol) were dissolved in CH₂Cl₂ (3 mL) and the mixture was stirred overnight. In the morning, saturated NaHCO₃ solution was added to the reaction, the organic phase was separated, washed with brine, and dried over Na₂SO₄. The crude material was purified by silica gel chromatography using a gradient of EtOAc/Hex (80 to 100%) to give (±)-tert-butyl {2-[({1-[(3-pyridin-3-ylpropanoyl)amino]-2,3-dihydro-1H-inden-5-yl}carbonyl)amino]phenyl}carbamate as a solid (80 mg, 58% yield). LC/MS [M+H] 501.1, RT 2.41 min. ¹H-NMR (DMSO-d6) δ 9.77 (s, 1H), 8.71 (br s, 1H), 8.44-8.45 (m, 1H), 8.39-8.41 (dd, 1H), 8.27 (d, 1H), 7.76 (s, 1H), 7.68-7.72 (m, 1H), 7.62-7.65 (ddd, 1H), 7.53-7.56 (dd, 1H), 7.46-7.48 (dd, 1H), 7.28-7.32 (ddd, 1H), 7.11-7.19 (m, 1H), 7.02 (d, 1H), 5.29 (q, 1H), 2.79-2.98 (m, 4H), 2.47-2.52 (m, 2H), 2.34-2.43 (m, 1H), 2.71-2.80 (m, 1H), 1.45 (s, 9H).

### Step 2: Preparation of Compound Example 122

The reaction was performed similarly as described in step 5 under compound example 6. LC/MS [M+H] 401.1, RT 1.20 min. ¹H-NMR (DMSO-d6) δ 9.59 (s, 1H), 8.45 (d, 1H), 8.40-8.41 (dd, 1H), 8.26 (d, 1H), 7.81 (s, 1H), 7.73 (d, 1H), 7.62-7.65 (ddd, 1H), 7.29-7.32 (dd, 1H), 7.13 (d, 1H), 6.98-6.99 (d, 1H), 6.92-6.97 (ddd, 1H), 6.74-6.77 (dd, 1H), 6.55-6.60 (ddd, 1H), 5.29 (q, 1H), 4.87 (s, 2H), 2.79-2.98 (m, 4H), 2.47-2.51 (m, 2H), 2.34-2.42 (m, 1H), 1.70-1.80 (m, 1H).

### Preparation Of Compound Example 123

### (±)-N-(2-aminophenyl)-1-{[(pyridin-3-yloxy)acetyl]amino}indane-5-carboxamide

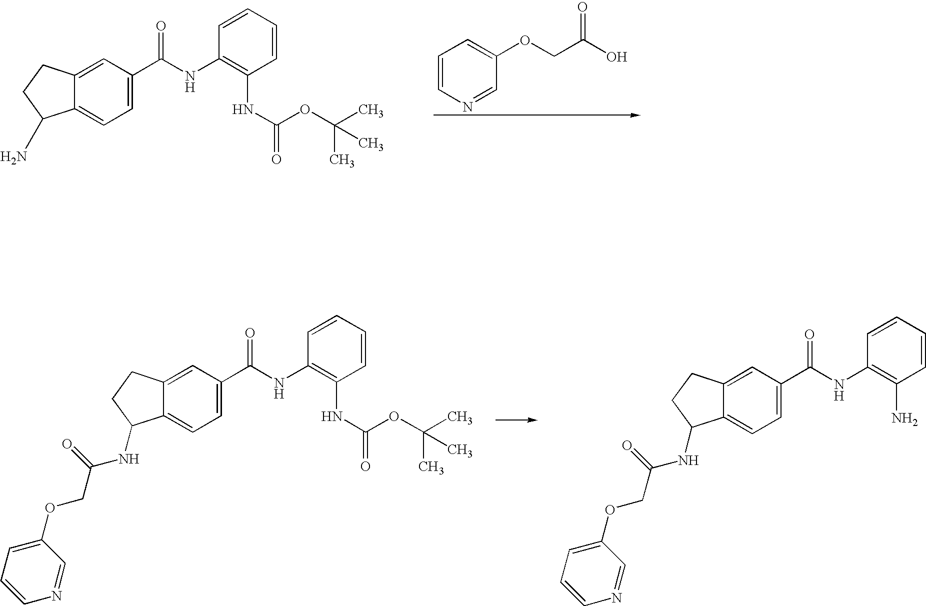

### Step 1: Preparation of Intermediate AG, (±)-tert-butyl (2-{[(1-{[(pyridin-3-yloxy)acetyl]amino}-2,3-dihydro-1H-inden-5-yl)carbonyl]amino}phenyl)carbamate

To a solution of 3-pyridyloxyacetic acid (63 mg, 0.41 mmol) was in CH₂Cl₂ (4 mL) at 0° C. was added few drops of DMF followed by oxalyl chloride (52 mg, 0.41 mmol). Gas evolution was seen upon addition. The reaction mixture was slowly warmed to rt and stirred for 1 h before it was concentrated and re-dissolved in CH₂Cl₂ (3 mL). To the above solution was added a solution of intermediate Q (100 mg, 0.27 mmol) and Et₃N (76 ul, 0.54 mmol) in CH₂Cl₂ (1 mL). After 1 h, saturated NaHCO₃ solution was added and the mixture was extracted with CH₂Cl₂ and the organic layer was washed with brine. The organic phase was collected, dried over Na₂SO₄, and concentrated under vacuum to give tert-butyl (2-{[(1-{[(pyridin-3-yloxy)acetyl]amino}-2,3-dihydro-1H-inden-5-yl)carbonyl]amino}phenyl)carbamate (90 mg, 66% yield). LC/MS [M+H] 503.1, RT 2.57 min. ¹H-NMR (DMSO-d6) δ 9.78 (s, 1H), 8.71 (br s, 1H), 8.60 (d, 1H), 8.31-8.32 (dd, 1H), 8.17-8.19 (dd, 1H), 7.79 (s, 1H), 7.75 (d, 1H), 7.54-7.56 (dd, 1H), 7.46-7.48 (dd, 1H), 7.32-7.40 (m, 2H), 7.24 (d, 1H), 7.11-7.19 (m, 2H), 5.43 (q, 1H), 4.68 (s, 2H), 2.97-3.05 (m, 1H), 2.84-2.92 (m, 1H), 2.40-2.47 (m, 1H), 1.92-2.02 (m, 1H), 1.45 (s, 9H).

### Step 2: Preparation of Compound Example 123

The reaction was performed similarly as described in step 5 under compound example 6. LC/MS [M+H] 403.1, RT 1.34 min. ¹H-NMR (DMSO-d6) δ 9.62 (s, 1H), 8.61 (d, 1H), 8.31-8.32 (dd, 1H), 8.17-8.18 (dd, 1H), 7.84 (s, 1H), 7.78 (d, 1H), 7.32-7.40 (m, 2H), 7.21 (d, 1H), 7.12-7.15 (dd, 1H), 6.92-6.96 (ddd, 1H), 6.75-6.77 (dd, 1H), 6.55-6.60 (ddd, 1H), 5.41 (q, 1H), 4.88 (br s, 2H), 4.68 (s, 2H), 2.97-3.04 (m, 1H), 2.84-2.92 (m, 1H), 2.39-2.47 (m, 1H), 1.92-2.02 (m, 1H).

The list of compound examples, their IUPAC names and LC-MS data are listed in table 1.

| Compound | | | LC-MS data |
|---|---|---|---|
| example | Structure | IUPAC Name | (RT in minutes) |
| 1 | 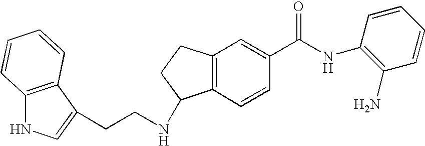 | N-(2-aminophenyl)-1-{[2-(1H-indol-3-yl)ethyl]amino}indane-5-carboxamide; | RT = 2.04 M + 1 = 411.2 |
| 2 | 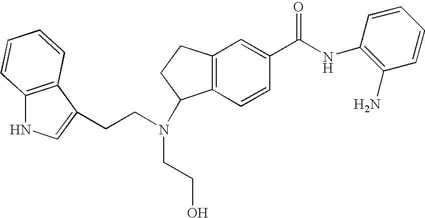 | N-(2-aminophenyl)-1-{(2-hydroxyethyl)[2-(1H-indol-3-yl)ethyl]amino}indane-5- carboxamide; | RT = 1.96 M + 1 = 455.2 |
| 3 | 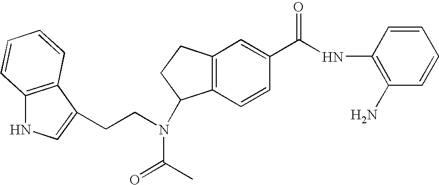 | 1-{acetyl[2-(1H-indol-3-yl)ethyl]amino}-N-(2-aminophenyl)indane-5-carboxamide; | RT = 2.61 M + 1 = 453.2 |
| 4 | 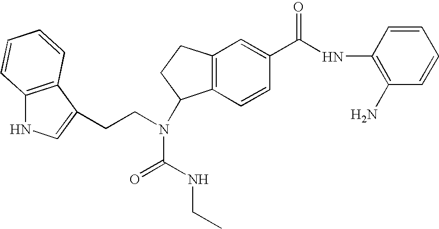 | N-(2-aminophenyl)-1-{[(ethylamino)carbonyl][2-(1H-indol-3-yl)ethyl]amino}indane-5- carboxamide; | RT = 2.69 M + 1 = 482.5 |
| 5 | 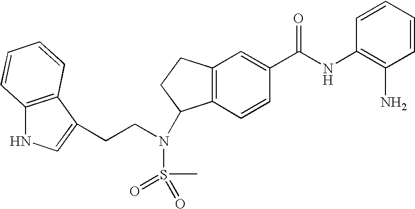 | N-{2-aminophenyl}-1-[[2-(1H-indol-3-yl)ethyl](methylsulfonyl)amino]indane-5- carboxamide; | R = 2.79 M + 1 = 489.1 |
| 6 | 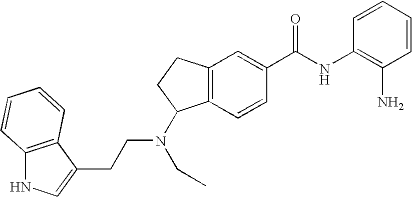 | N-(2-aminophenyl)-1-[ethyl[2-(1H-indol-3-yl)ethyl]indane-5-carboxamide; | RT = 2.10 M + 1 = 439.2 |
| 7 | 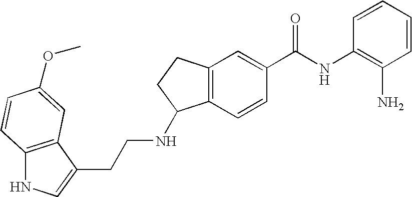 | N-(2-aminophenyl)-1-{[2-(5-methoxy-1H-indol-3-yl)ethyl]amino}indane-5- carboxamide; | RT = 2.01 M + 1 = 441.1 |
| 8 | 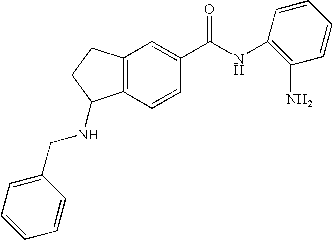 | N-(2-aminophenyl)-1-(benzylamino)indane-5-carboxamide; | RT = 1.68 M + 1 = 358.0 |
| 9 | 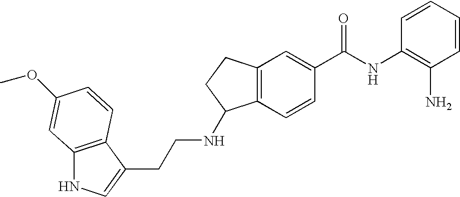 | N-(2-aminophenyl)-1-{[2-(6-methoxy-1H-indol-3-yl)ethyl]amino}indane-5- carboxamide; | RT = 2.02 M + 1 = 441.2 |
| 10 | 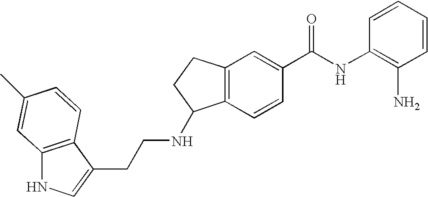 | N-(2-aminophenyl)-1-{[2-(6-methyl-1H-indol-3-yl)ethyl]amino}indane-5- carboxamide; | RT = 2.15 M + 1 = 425.2 |
| 11 | 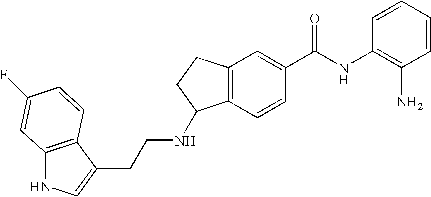 | N-(2-aminophenyl)-1-{[2-(6-fluoro-1H-indol-3-yl)ethyl]amino}indane-5- carboxamide; | RT = 2.11 M + 1 = 429.2 |
| 12 | 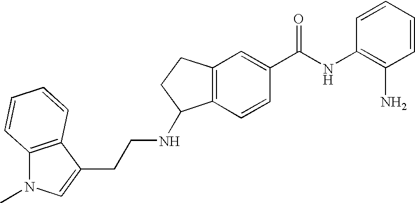 | N-(2-aminophenyl)-1-{[2-(1-methyl-1H-indol-3-yl)ethyl]amino}indane-5- carboxamide; | RT = 2.02 M + 1 = 425.2 |
| 13 | 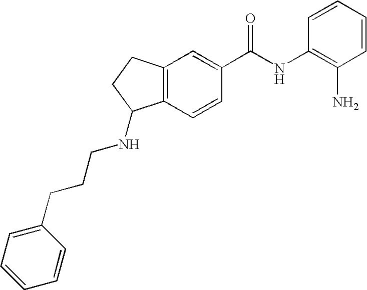 | N-(2-aminophenyl)-1-{(3-phenylpropyl)amino]indane-5-carboxamide; | RT = 2.06 M + 1 = 386.0 |
| 14 | 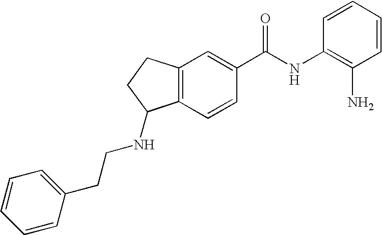 | N-(2-aminophenyl)-1-[(2-phenylethyl)amino]indane-5-carboxamide; | RT = 1.90 M + 1 = 372.0 |
| 15 | 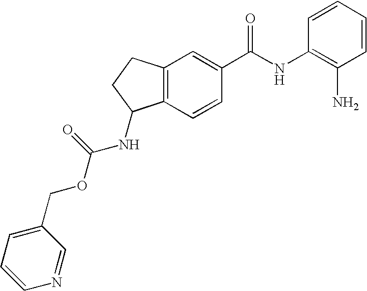 | pyridin-3-ylmethyl (5-{[(2-aminophenyl)amino]carbonyl}-2,3-dihydro-1H-inden-1- yl)carbamate; | RT: 1.44 M + 1 = 403.2 |
| 16 | 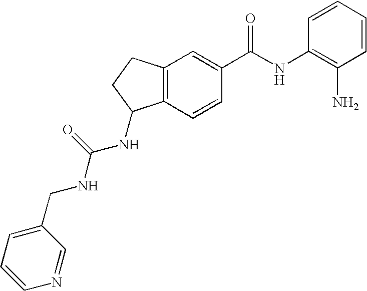 | N-(2-aminophenyl)-1-({[(pyridin-3-ylmethyl)amino]carbonyl}amino)indane-5- carboxamide; | RT: 2.09 M + 1 = 402.2 |
| 17 | 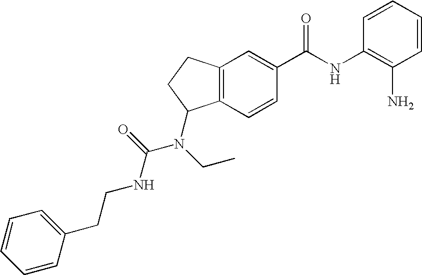 | N-(2-aminophenyl)-1-{ethyl{[(2-phenylethyl)amino]carbonyl}amino)indane-5- carboxamide; | RT = 2.69 M + 1 = 443.2 |
| 18 | 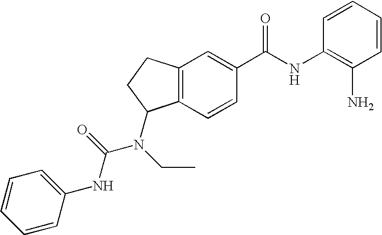 | N-(2-aminophenyl)-1-[(anilinecarbonyl)(ethyl)amino]indane-5-carboxamide; | RT = 2.59 M + 1 = 415.2 |
| 19 | 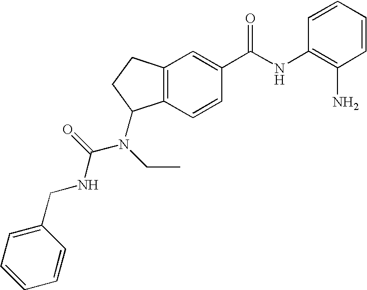 | N-(2-aminophenyl)-1-[[(benzylamino)carbonyl](ethyl)amino]indane-5-carboxamide | RT = 2.58 M + 1 = 429.2 |
| 20 | 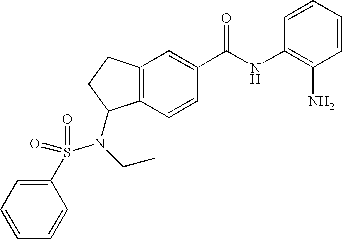 | N-(2-aminophenyl)-1-[ethyl(phenylsulfonyl)amino]indane-5-carboxamide; | RT = 2.85 M + 1 = 436.2 |
| 21 | 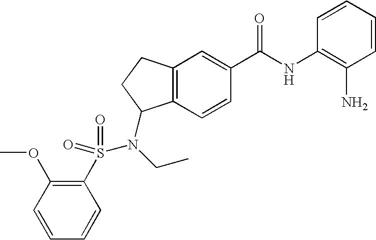 | N-(2-aminophenyl)-1-{ethyl[(2-methoxyphenyl)sulfonyl]amino)indane-5- carboxamide; | RT = 2.79 M + 1 = 466.2 |
| 22 | 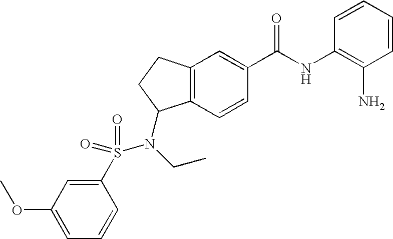 | N-(2-aminophenyl)-1-{ethyl[(3-methoxyphenyl)sulfonyl]amino}indane-5- carboxamide; | RT = 2.92 M + 1 = 466.1 |
| 23 | 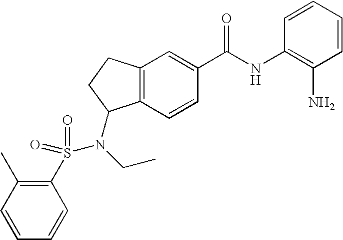 | N-(2-aminophenyl)-1-{ethyl[(2-methylphenyl)sulfonyl]amino}indane-5- carboxamide; | RT = 2.98 M + 1 = 450.2 |
| 24 | 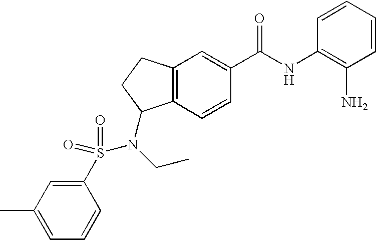 | N-(2-aminophenyl)-1-[ethyl[(3-methylphenyl)sulfonyl]amino}indane-5- carboxamide; | RT = 2.99 M + 1 = 450.2 |
| 25 | 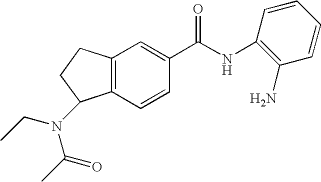 | 1-[acetyl(ethyl)amino]-N-{2-aminophenyl)indane-5-carboxamide; | RT = 2.08 M + 1 = 338.2 |
| 26 | 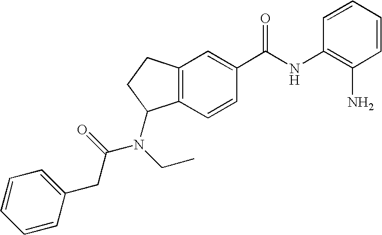 | N-(2-aminophenyl)-1-[ethyl(phenylacetyl)amino]indane-5-carboxamide; | RT = 2.64 M + 1 = 414.3 |
| 27 | 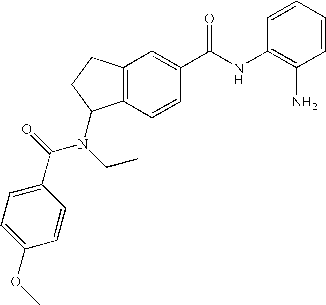 | N-{2-aminophenyl)-1-[ethyl(4-methoxybenzoyl)amino]indane-5-carboxamide; | RT = 2.57 M + 1 = 430.3 |
| 28 | 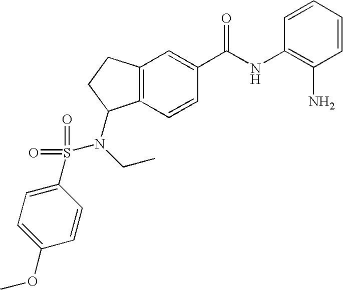 | N-(2-aminophenyl)-1-{ethyl[(4-methoxyphenyl)sulfonyl]amino}indane-5- carboxamide; | RT = 2.90 M + 1 = 466.2 |
| 29 | 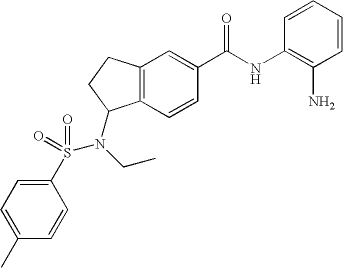 | N-(2-aminophenyl)-1-(ethyl[(4-methylphenyl)sulfonyl]amino}indane-5- carboxamide | RT = 3.00 M + 1 = 450.2 |
| 30 | 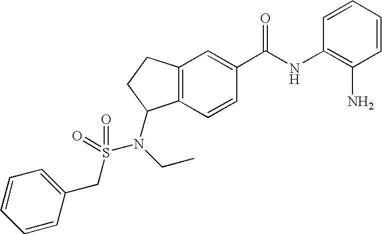 | N-(2-aminophenyl)-1-[(benzylsulfonyl)(ethyl)amino]indane-5-carboxamide; | RT = 2.88 M + 1 = 450.2 |
| 31 | 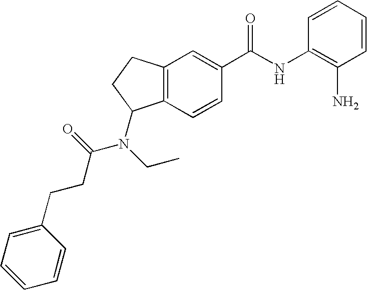 | N-(2-aminophenyl)-1-[ethyl(3-phenylpropanyl)amino]indane-5-carboxamide; | RT = 2.80 M + 1 = 428.4 |
| 32 | 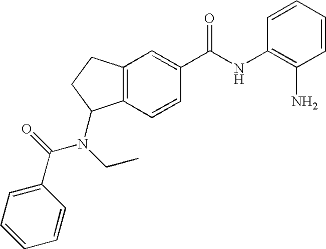 | N-(2-aminophenyl)-1-[benzyl(ethyl)amino]indane-5-carboxamide; | RT = 2.54 M + 1 = 400.3 |
| 33 | 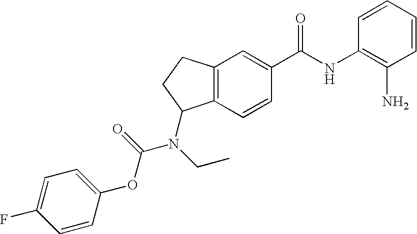 | 4-fluorophenyl (5-{[(2-aminophenyl)amino]carbonyl}-2,3-dihydro-1H-inden-1- yl)ethylcarbamate; | RT = 2.96 M + 1 = 434.3 |
| 34 | 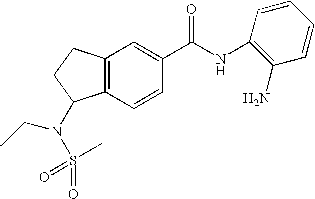 | N-(2-aminophenyl)-1-[ethyl(methylsulfonyl)amino]indane-5-carboxamide; | RT = 2.26 M + 1 = 374.2 |
| 35 | 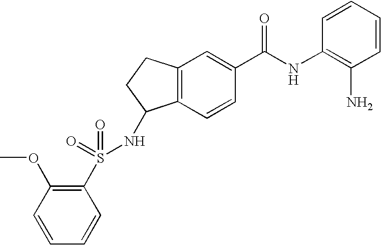 | N-(2-aminophenyl)-1-{[(2-methoxyphenyl)sulfonyl]amino}indane-5-carboxamide; | RT: 2.53 M + 1 = 438.1 |
| 36 | 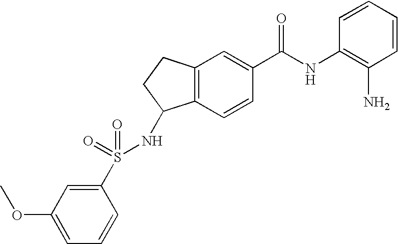 | N-[2-aminophenyl)-1-{[(3-methoxyphenyl)sulfonyl]amino}indane-5-carboxamide; | RT: 2.61 M + 1 = 438.1 |
| 37 | 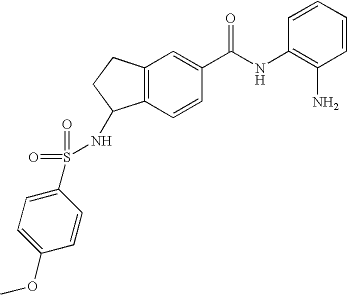 | N-(2-aminophenyl)-1-{[(4-methoxyphenyl)sulfonyl]amino}indane-5-carboxamide; | RT: 2.58 M + 1 = 438.1 |
| 38 | 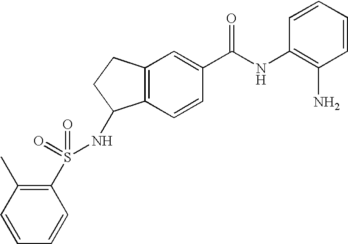 | N-(2-aminophenyl)-1-{[(2-methylphenyl)sulfonyl]amino}indane-5-carboxamide; | RT: 2.67 M + 1 = 422.1 |
| 39 | 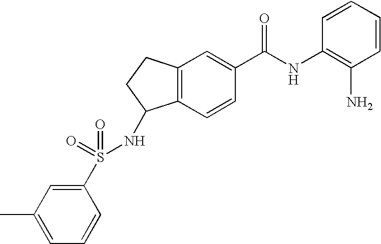 | N-(2-aminophenyl)-1-{[(3-methylphenyl)sulfonyl]amino}indane-5-carboxamide; | RT: 2.68 M + 1 = 422.1 |
| 40 | 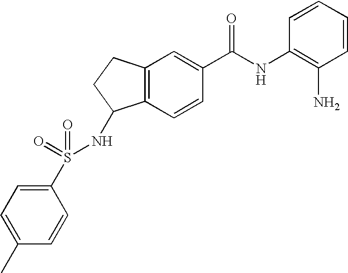 | N-(2-aminophenyl)-1-{[(4-methylphenyl)sulfonyl]amino}indane-5-carboxamide; | RT: 2.68 M + 1 = 422.1 |
| 41 | 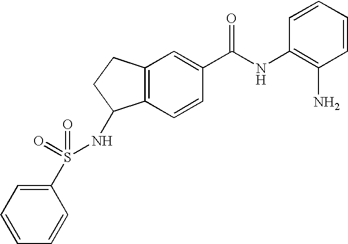 | N-(2-aminophenyl)-1-[(phenylsulfonyl)amino]indane-5-carboxamide; | RT: 2.52 M + 1 = 406.1 |
| 42 | 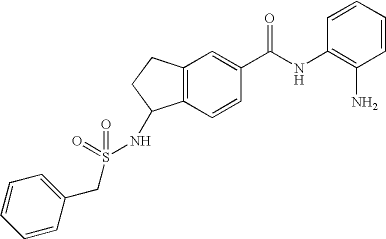 | N-(2-aminophenyl)-1-[(benzylsulfonyl)amino]indane-5-carboxamide; | RT = 2.55 M + 1 = 422.1 |
| 43 | 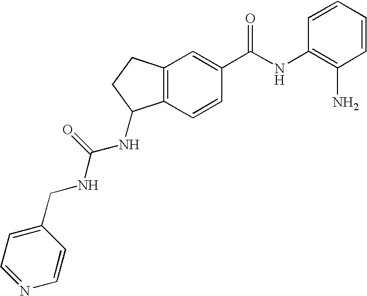 | N-(2-aminophenyl)-1-({[(pyridin-4-ylmethyl)amino]carbonyl}amino)indane-5- carboxamide; | RT: 1.10 M + 1 = 402.2 |
| 44 | 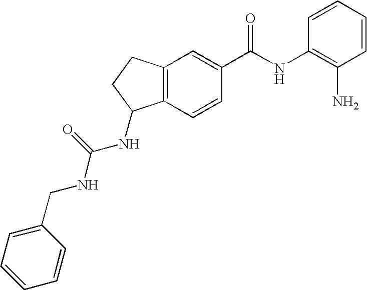 | N-(2-aminophenyl)-1-{[(benzylamino)carbonyl]amino)indane-5-carboxamide; | RT: 2.34 M + 1 = 401.2 |
| 45 | 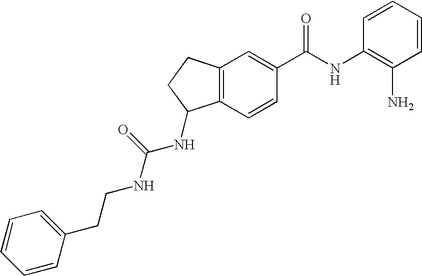 | N-(2-aminophenyl)-1-({[(2-phenylethyl)amino]carbonyl}amino)indane-5- carboxamide; | RT: 2.46 M + 1 = 415.1 |
| 46 | 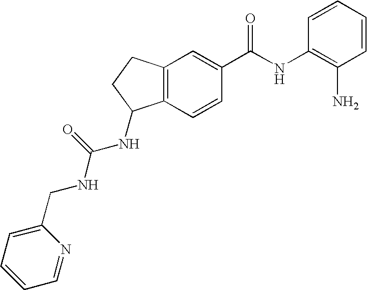 | N-(2-aminophenyl)-1-({[(pyridin-2-ylmethyl)amino]carbonyl}amino)indane-5- carboxamide; | RT: 1.12, M + 1 = 402.2 |
| 47 | 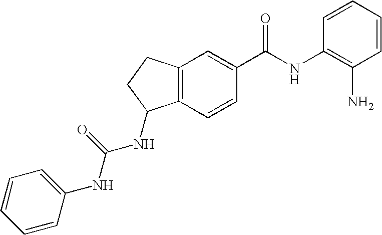 | N-(2-aminophenyl)-1-[(anilinocarbonyl)amino]indane-5-carboxamide; | RT: 2.39 M + 1 = 387.3 |
| 48 | 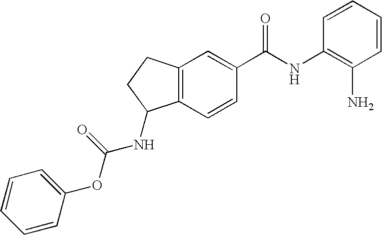 | phenyl (5-{[(2-aminophenyl)amino]carbonyl}-2,3-dihydro-1H-inden-1-yl)carbamate; | RT: 2.57 M + 1 = 388.2 |
| 49 | 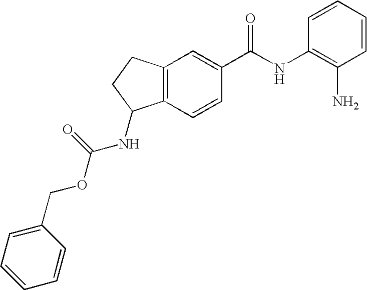 | benzyl (5-{[(2-aminophenyl)amino]carbonyl}-2,3-dihydro-1H-inden-1-yl)carbamate; | RT: 2.70 M + 1 = 402.3 |
| 50 | 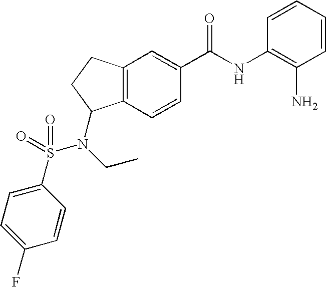 | N-(2-aminophenyl)-1-{ethyl[(4-fluorophenyl)sulfonyl]amino}indane-5-carboxamide; | RT = 2.95 M + 1 = 454.0 |
| 51 | 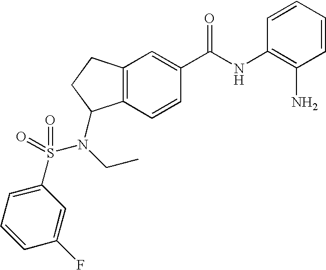 | N-(2-aminophenyl)-1-(ethyl[(3-fluorophenyl)sulfonyl]amino)indane-5-carboxamide; | RT = 2.97 M + 1 = 454.0 |
| 52 | 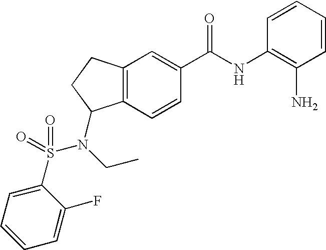 | N-(2-aminophenyl)-1-{ethyl[(2-fluorophenyl)sulfonyl]amino}indane-5-carboxamide; | RT = 2.92 M + 1 = 454.0 |
| 53 | 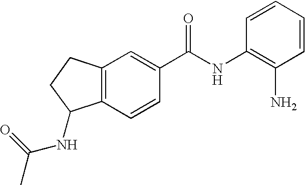 | 1-(acetylamino)-N-(2-aminophenyl)indane-5-carboxamide; | RT: 1.40 M + 1 = 310.1 |
| 54 | 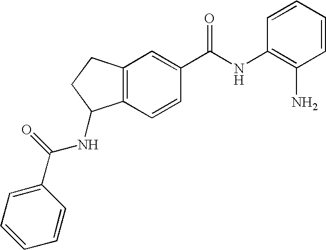 | N-(2-aminophenyl)-1-(benzoylamino)indane-5-carboxamide; | RT: 2.34 M + 1 = 372.1 |
| 55 | 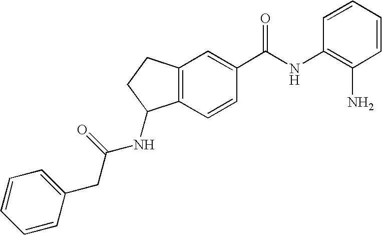 | N-(2-aminophenyl)-1-[(phenylacetyl)amino]indane-5-carboxamide; | RT: 2.38 M + 1 = 386.2 |
| 56 | 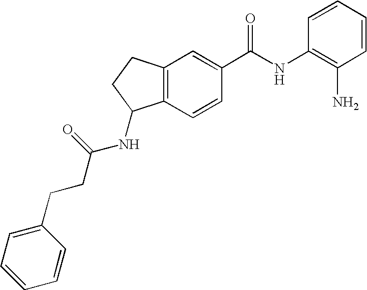 | N-(2-aminophenyl)-1-[(3-phenylpropanoyl)amino]indane-5-carboxamide; | RT: 2.48 M + 1 = 400.2 |
| 57 | 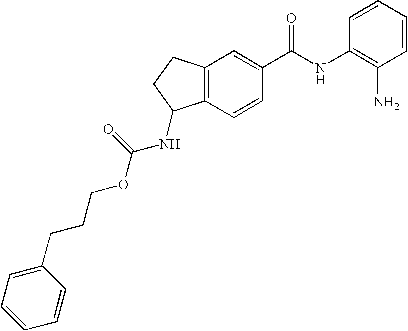 | 3-phenylpropyl (5-{[(2-aminophenyl)amino]carbonyl}-2,3-dihydro-1H-inden-1- yl)carbamate; | RT: 2.97 M + 1 = 430.2 |
| 58 | 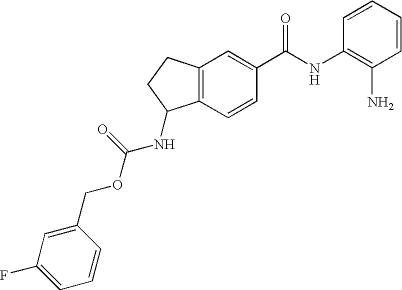 | 3-fluorobenzyl (5-{[(2-aminophenyl)amino]carbonyl}-2,3-dihydro-1H-inden-1- yl)carbamate; | RT: 2.80 M + 1 = 420.2 |
| 59 | 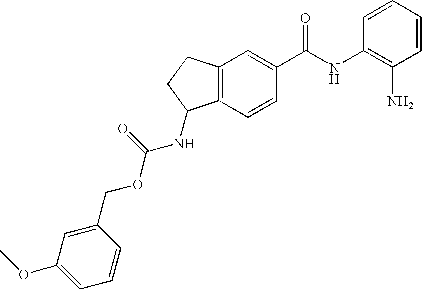 | 3-methoxybenzyl (5-{[(2-aminophenyl)amino]carbonyl}-2,3-dihydro-1H-inden-1- yl)carbamate; | RT: 2.76 M + 1 = 432.2 |
| 60 | 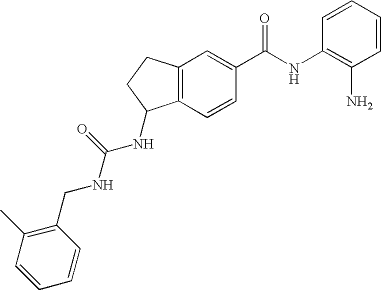 | N-(2-aminophenyl)-1-({[(2-methylbenzyl)amino]carbonyl}amino)indane-6- carboxamide; | RT: 2.52 M + 1 = 415.1 |
| 61 | 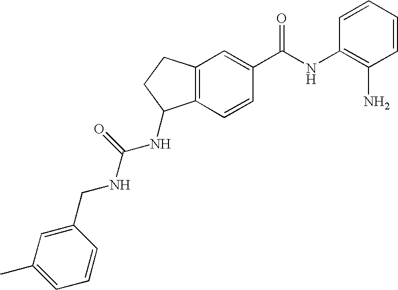 | N-(2-aminophenyl)-1-({[(3-methylbenzyl)amino]carbonyl}amino)indane-5- carboxamide; | RT: 2.55 M + 1 = 415.1 |
| 62 | 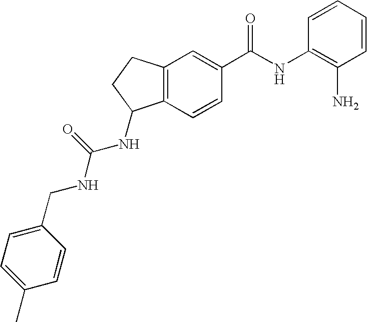 | N-(2-aminophenyl)-1-({[(4-methylbenzyl)amino]carbonyl}amino)indane-5- carboxamide; | RT: 2.56 M + 1 = 415.0 |
| 63 | 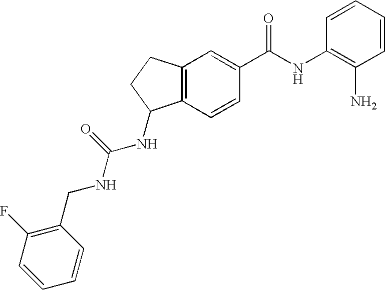 | N-(2-aminophenyl)-1-({[(2-fluorobenzyl)amino]carbonyl}amino)indane-5- carboxamide; | RT: 2.44 M + 1 = 419.1 |
| 64 | 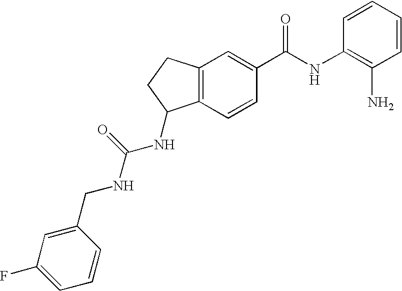 | N-(2-aminophenyl)-1-({[(3-fluorobenzyl)amino]carbonyl}amino)indane-5- carboxamide; | RT: 2.46 M + 1 = 419.0 |
| 65 | 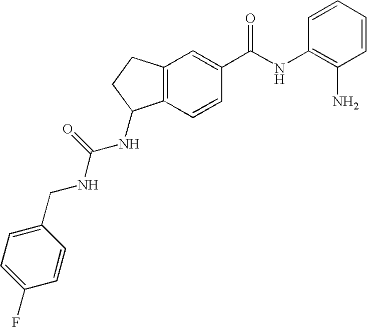 | N-(2-aminophenyl)-1-({[(4-fluorobenzyl)amino]carbonyl}amino)indane-5- carboxamide; | RT: 2.46 M + 1 = 419.0 |
| 66 | 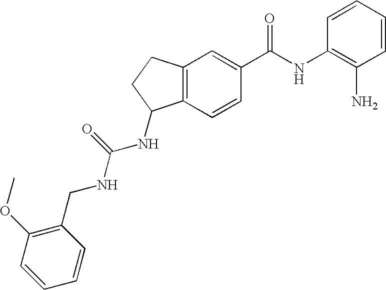 | N-(2-aminophenyl)-1-({[(2-methoxybenzyl)amino]carbonyl}amino)indane-5- carboxamide; | RT: 2.47 M + 1 = 431.0 |
| 67 | 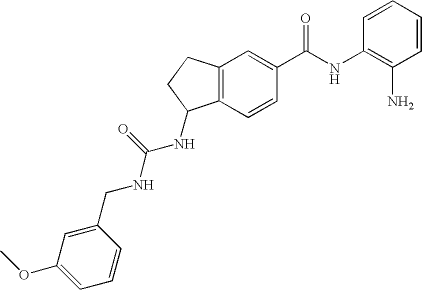 | N-(2-aminophenyl)-1-({[(3-methoxybenzyl)amino]carbonyl}amino)indane-5- carboxamide; | RT: 2.41 M + 1 = 431.0 |
| 68 | 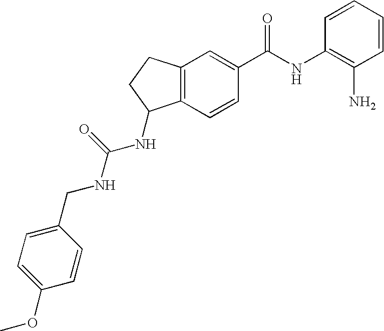 | N-(2-aminophenyl)-1-({[(4-methoxybenzyl)amino]carbonyl}amino)indane-5- carboxamide; | RT: 2.39 M + 1 = 431.1 |
| 69 | 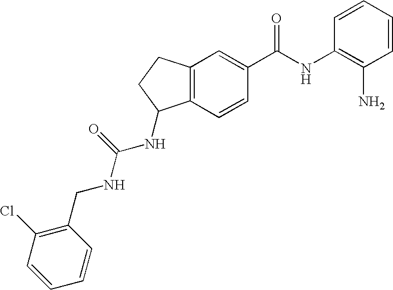 | N-(2-aminophenyl)-1-({[(2-chlorobenzyl)amino]carbonyl}amino)indane-5- carboxamide; | RT: 2.56 M + 1 = 435.0 |
| 70 | 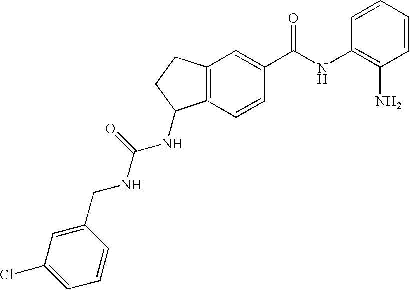 | N-(2-aminophenyl)-1-({[(3-chlorobenzyl)amino]carbonyl]amino)indane-5- carboxamide; | RT: 2.60 M + 1 = 435.0 |
| 71 | 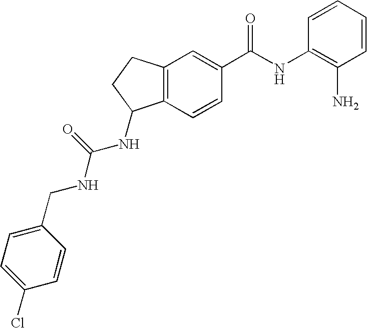 | N-(2-aminophenyl)-1-({[(4-chlorobenzyl)amino]carbonyl}amino)indane-5- carboxamide; | RT: 2.61 M + 1 = 435.0 |
| 72 | 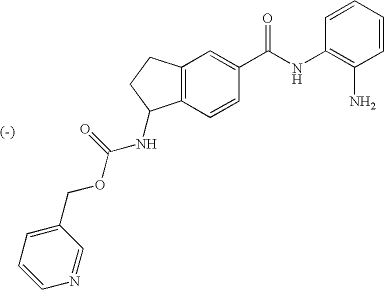 | (−)-pyridin-3-ylmethyl ((1R)-5-{[(2-aminophenyl)amino]carbonyl}-2,3-dihydro-1H- inden-1-yl)carbamate; | RT: 2.25 M + 1 = 403.1 |
| 73 | 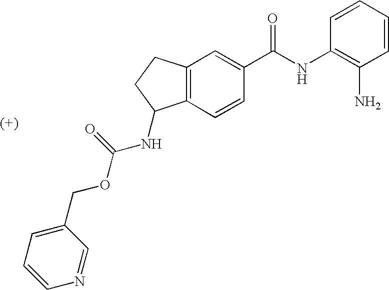 | (+)-pyridin-3-ylmethyl ((1S)-5-{[(2-aminophenyl)amino]carbonyl}-2,3-dihydro-1H- inden-1-yl)carbamate; | RT: 2.25 M + 1 = 403.0 |
| 74 | 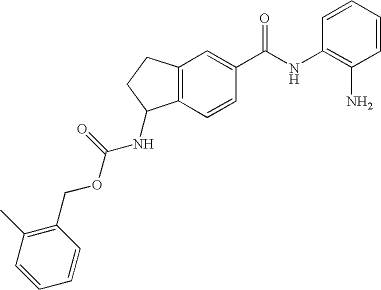 | 2-methylbenzyl (5-{[(2-aminophenyl)amino]carbonyl}-2,3-dihydro-1H-inden-1- yl)carbamate; | RT: 2.86 M + 1 = 416.1 |
| 75 | 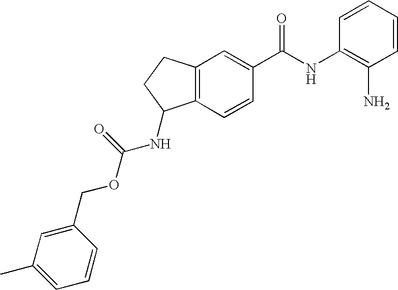 | 3-methylbenzyl (5-{[(2-aminophenyl)amino]carbonyl}-2,3-dihydro-1H-inden-1- yl)carbamate; | RT: 2.89 M + 1 = 416.0 |
| 76 | 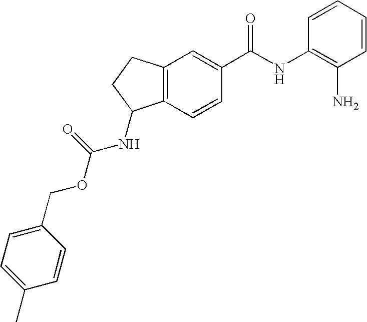 | 4-methylbenzyl (5-{[(2-aminophenyl)amino]carbonyl}-2,3-dihydro-1H-inden-1- yl)cabamate; | RT: 2.90 M + 1 = 416.1 |
| 77 | 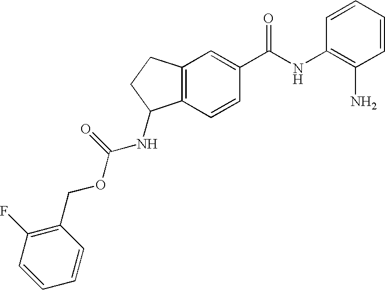 | 2-fluorobenzyl (5-{[(2-aminophenyl)amino]carbonyl}-2,3-dihydro-1H-inden-1- yl)carbamate; | RT: 2.77 M + 1 = 420.0 |
| 78 | 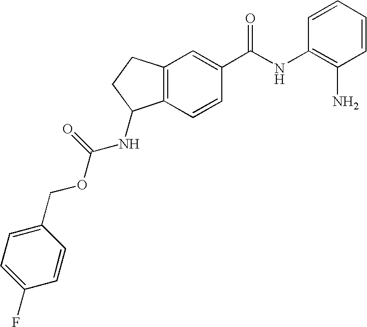 | 4-fluorobenzyl (5-{[(2-aminophenyl)amino]carbonyl}-2,3-dihydro-1H-inden-1- yl)carbamate; | RT: 2.78 M + 1 = 420.0 |
| 79 | 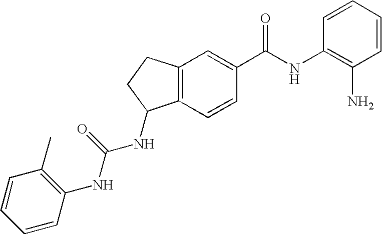 | N-(2-aminophenyl)-1-({[(2-methylphenyl)amino]carbonyl}amino)indane-5- carboxamide; | RT: 2.47 M + 1 = 401.0 |
| 80 | 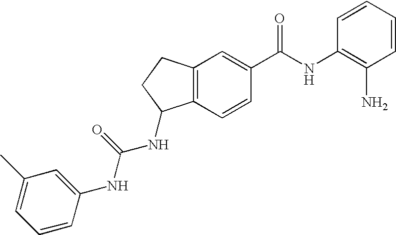 | N-(2-aminophenyl)-1-({[(3-methylphenyl)amino]carbonyl}amino)indane-5- carboxamide; | RT: 2.59 M + 1 = 401.0 |
| 81 | 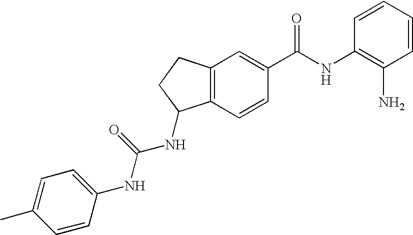 | N-(2-aminophenyl)-1-({[(4-methylphenyl)amino]carbonyl}amino)indane-5- carboxamide; | RT: 2.58 M + 1 = 401.0 |
| 82 | 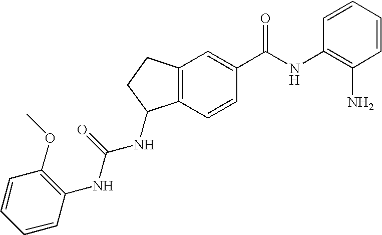 | N-(2-aminophenyl)-1-({[(2-methoxyphenyl)amino]carbonyl}amino)indane-5- carboxmaide; | RT: 2.54 M + 1 = 417.0 |
| 83 | 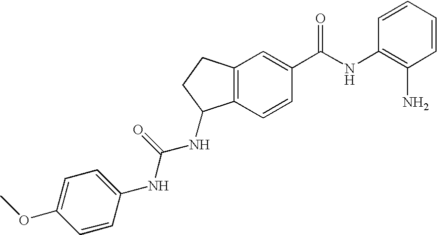 | N-(2-aminophenyl)-1-({[(4-methoxyphenyl)amino]carbonyl}amino)indane-5- carboxamide; | RT: 2.39 M + 1 = 417.0 |
| 84 | 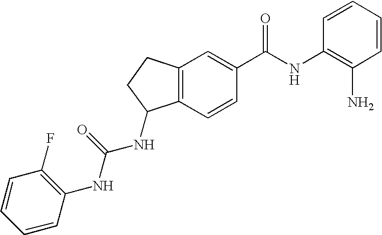 | N-(2-amionphenyl)-1-({[(2-fluorophenyl)amino]carbonyl}amino)indane-5- carboxamide; | RT: 2.51 M + 1 = 405.0 |
| 85 | 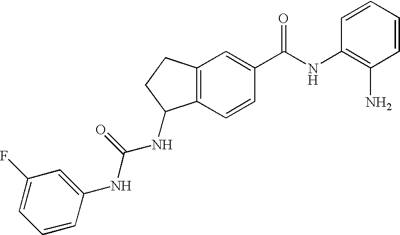 | N-(2-aminophenyl)-1-({[(3-fluorophenyl)amino]carbonyl}amino)indane-5- carboxamide; | RT: 2.58 M + 1 = 405.0 |
| 86 | 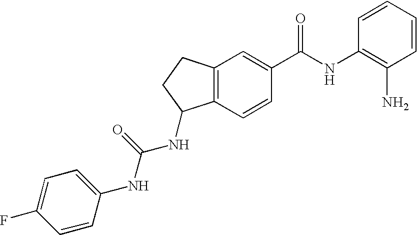 | N-(2-aminophenyl)-1-({[(4-fluorophenyl)amino]carbonyl}amino)indene-5- carboxamide; | RT: 2.50 M + 1 = 405.0 |
| 87 | 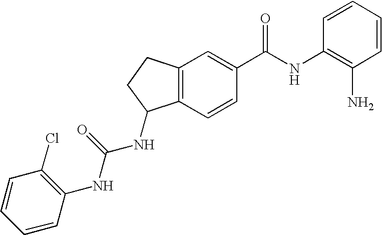 | N-(2-aminophenyl)-1-({[2-chlorophenyl)amino]carbonyl}amino)indane-5- carboxamide; | RT: 2.65 M + 1 = 421.0 |
| 88 | 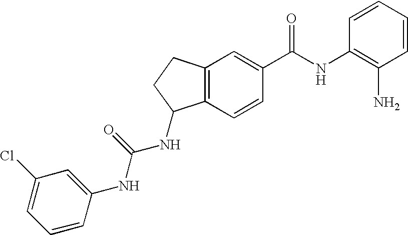 | N-(2-aminophenyl)-1-({[(3-chlorophenyl)amino]carbonyl}amino)indane-5- carboxamide; | RT: 2.75 M + 1 = 421.0 |
| 89 | 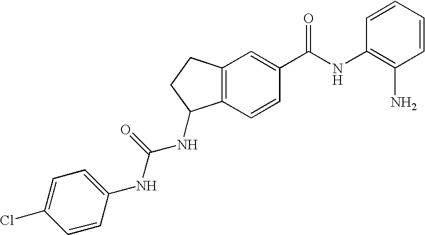 | N-(2-aminophenyl)-1-({[(4-chlorophenyl)amino]carbonyl}amino)indane-5- carboxamide; | RT: 2.72 M + 1 = 421.0 |
| 90 | 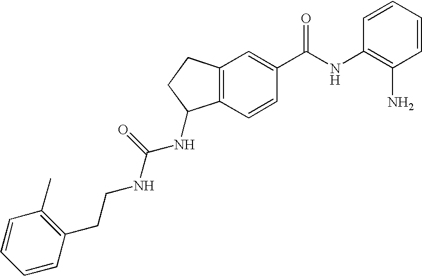 | N-(2-aminophenyl)-1-[({[2-(2-methylphenyl)ethyl]amino}carbonyl)amino]indane-5- carboxamide; | RT: 2.65 M + 1 = 429.1 |
| 91 | 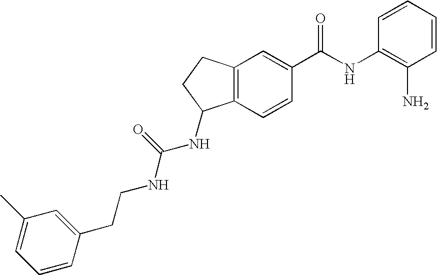 | N-(2-aminophenyl-1-[({[2-(3-methylphenyl)ethyl]amino}carbonyl)amino]indane-5- carboxamide; | RT: 2.67 M + 1 = 429.1 |
| 92 | 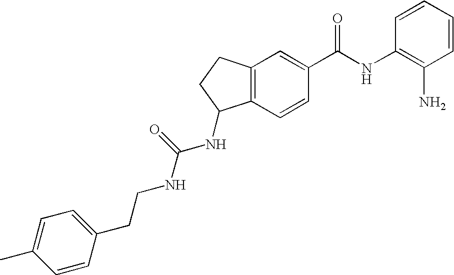 | N-(2-aminophenyl)-1-[({[2-(4-methylphenyl)ethyl]amino}carbonyl)amino]indane-5- carboxamide; | RT: 2.68 M + 1 = 429.1 |
| 93 | 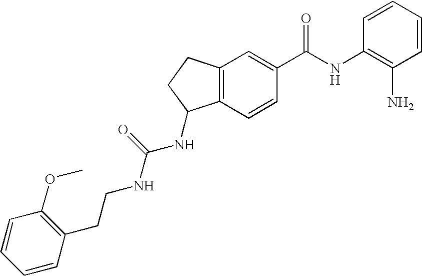 | N-(2-aminophenyl)-1-[({[2-(2-methoxyphenyl)ethyl]amino}carbonyl)amino]indane-5- carboxamide; | RT: 2.59 M + 1 = 445.1 |
| 94 | 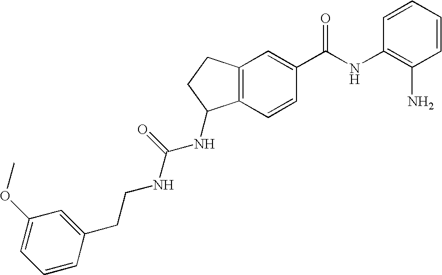 | N-(2-aminophenyl-1-[({[2-(3-methoxyphenyl)ethyl]amino}carbonyl)amino]indane-5- carboxamide; | RT: 2.54 M + 1 = 445.1 |
| 95 | 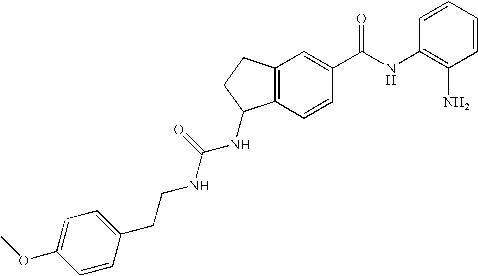 | N-(2-aminophenyl)-1-[({[2-(4-methoxyphenyl)ethyl]amino}carbonyl)amino]indane-5- carboxamide; | RT: 2.52 M + 1 = 445.0 |
| 96 | 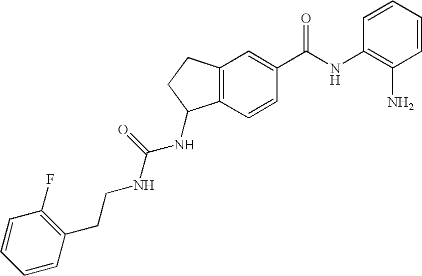 | N-(2-aminophenyl)-1-[({[2-(2-fluorophenyl)ethyl]amino}carbonyl)amino]indane-5- carboxamide; | RT: 2.56 M + 1 = 433.1 |
| 97 | 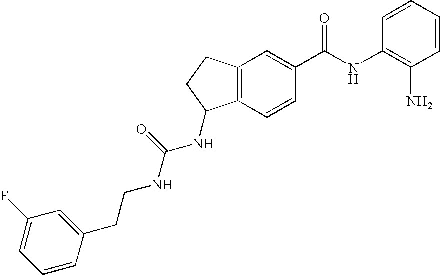 | N-(2-aminophenyl)-1-[({[2-(3-fluorophenyl)ethyl]amino}carbonyl)amino]indane-5- carboxamide; | RT: 2.57 M + 1 = 433.0 |
| 98 | 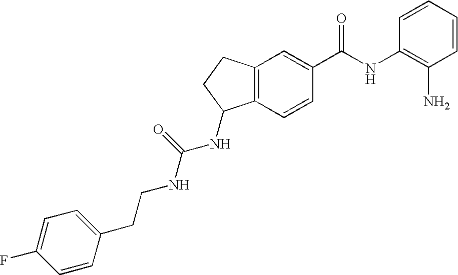 | N-(2-aminophenyl)-1-[({[2-(4-fluorophenyl)ethyl]amino}carbonyl)amino]indane-5- carboxamide; | RT: 2.58 M + 1 = 433.1 |
| 99 | 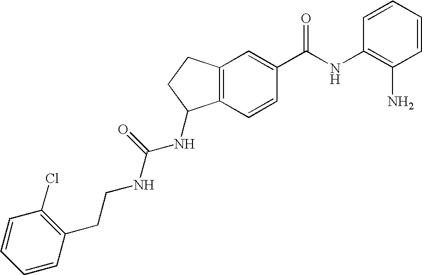 | N-(2-aminophenyl)-1-[({[2-(2-chlorophenyl)ethyl]amino}carbonyl)amino]indane-5- carboxamide; | RT: 2.68 M + 1 = 449.0 |
| 100 | 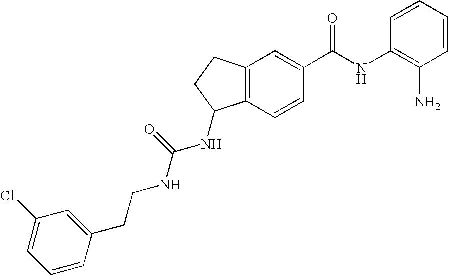 | N-(2-aminophenyl)-1-[({[2-(3-chlorophenyl)ethyl]amino}carbonyl)amino]indane-5- carboxamide; | RT: 2.73 M + 1 = 449.0 |
| 101 | 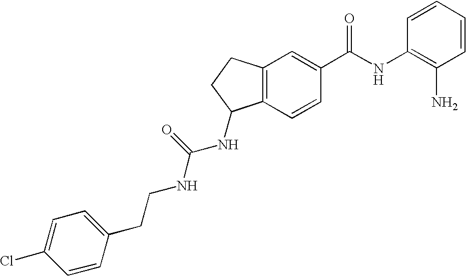 | N-(2-aminophenyl)-1-[({[2-(4-chlorophenyl)amino}carbonyl)amino]indane-5- carboxamide; | RT: 2.71 M + 1 = 449.0 |
| 102 | 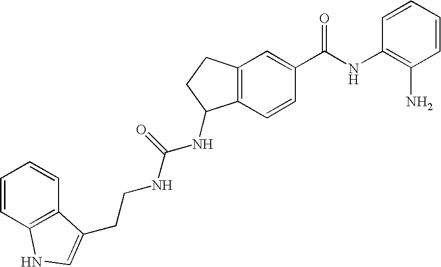 | N-(2-aminophenyl)-1-[({[2-(1H-indol-3-yl)ethyl]amino}carbonyl)amino]indane-5- carboxamide; | RT: 2.54 M + 1 = 454.1 |
| 103 | 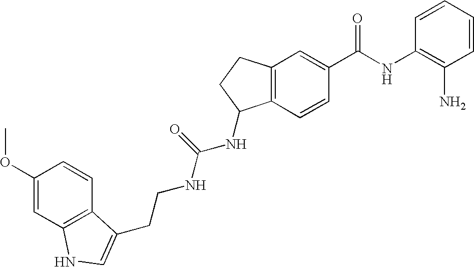 | N-(2-aminophenyl)-1-[({[2-(6-methoxy-1H-indol-3- yl)ethyl]amino}carbonyl)amino]indane-5-carboxamide; | RT: 2.51 M + 1 = 484.1 |
| 104 | 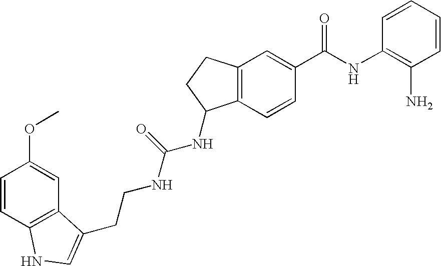 | N-(2-aminophenyl)-1-[({[2-(5-methoxy-1H-indol-3- yl)ethyl]amino}carbonyl)amino]indane-5-carboxamide; | RT: 2.48 M + 1 = 484.0 |
| 105 | 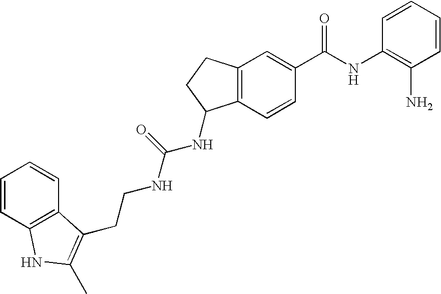 | N-(2-aminophenyl)-1-[({[2-(2-methyl-1H-indol-3- yl)ethyl]amino}carbonyl)amino]indane-5-carboxamide; | RT: 2.60 M + 1 = 468.0 |
| 106 | 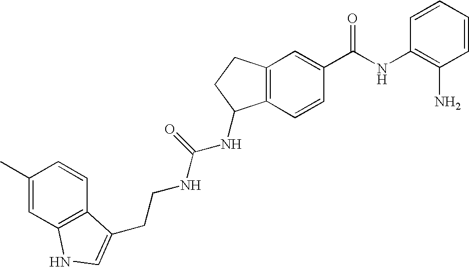 | N-(2-aminophenyl)-1-[({[2-(6-methyl-1H-indol-3- yl)ethyl]amino}carbonyl)amino]indane-5-carboxamide; | RT: 2.67 M + 1 = 468.1 |
| 107 | 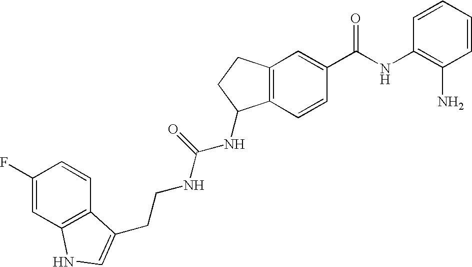 | N-(2-aminophenyl)-1-[({[2-(6-fluoro-1H-indol-3- yl)ethyl]amino}carbonyl)amino]indane-5-carboxamide; | RT: 2.62 M + 1 = 472.1 |
| 108 | 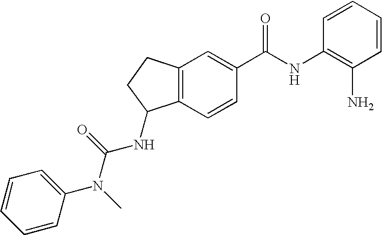 | N-(2-aminophenyl)-1-({[methyl(phenyl)amino]carbonyl}amino)indane-5- carboxamide; | RT: 2.46 M + 1 = 401.0 |
| 109 | 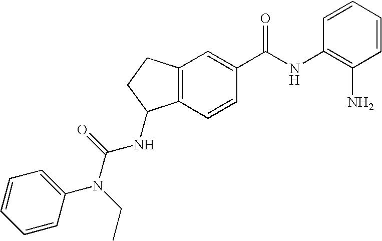 | N-(2-aminophenyl)-1-({[ethyl(phenyl)amino]crbonyl}amino)indane-5-carboxamide; | RT: 2.60 M + 1 = 415.0 |
| 110 | 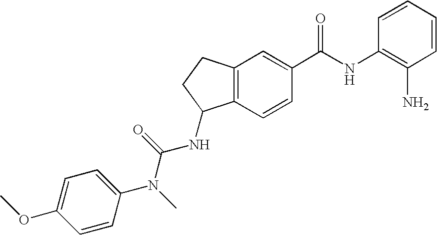 | N-(2-aminophenyl)-1-({[(4-methoxyphenyl)(methyl)amino]carbonyl}amino)indane-5- carboxamide; | RT: 2.50 M + 1 = 431.0 |
| 111 | 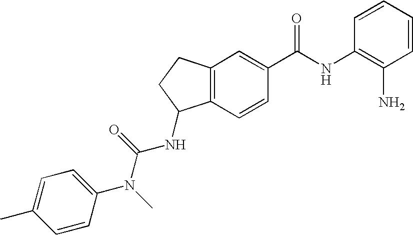 | N-(2-aminophenyl)-1-({[methyl(4-methyphenyl)amino]carbonyl}amino)indane-5- carboxamide; | RT: 2.64 M + 1 = 415.0 |
| 112 | 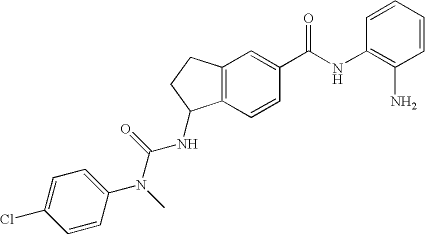 | N-(2-aminophenyl)-1-({[(4-chlorophenyl)(methyl)amino]carbonyl}amino)indane-5- carboxamide; | RT: 2.70 M + 1 = 435.0 |
| 113 | 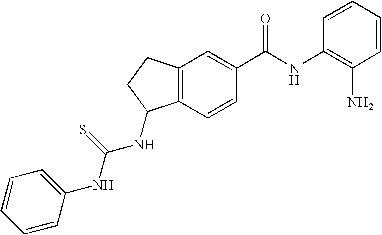 | N-(2-aminophenyl)-1-[(anilinocarbonothioyl)amino]indane-5-carboxamide; | RT: 2.58 M + 1 = 403.0 |
| 114 | 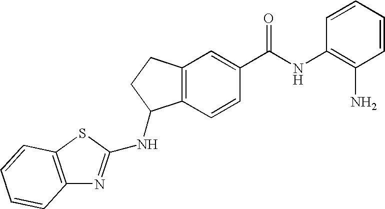 | N-(2-aminophenyl)-1-(1,3-benzothiazol-2-ylamino)indane-5-carboxamide; | RT: 2.37 M + 1 = 401.0 |
| 115 | 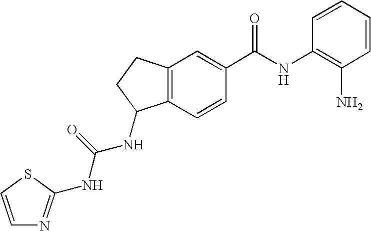 | N-(2-aminophenyl)-1-{[(1,3-thiazol-2-ylamino)carbonyl]amino}indane-5- carboxamide; | RT: 2.20 M + 1 = 394.0 |
| 116 | 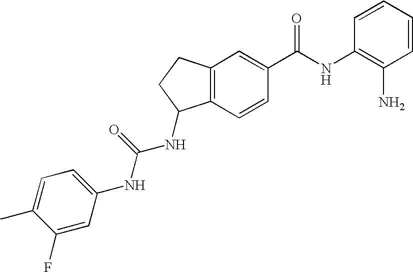 | N-(2-aminophenyl)-1-({[(3-fluoro-4-methylphenyl)amino]carbonyl}amino)indane-5- carboxamide; | RT: 2.75 M + 1 = 419.1 |
| 117 | 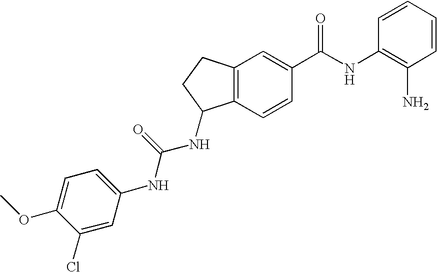 | N-(2-aminophenyl)-1-({[(3-chloro-4-methoxyphenyl)amino]carbonyl}amino)indane- 5-carboxamide; | RT: 2.66 M + 1 = 451.1 |
| 118 | 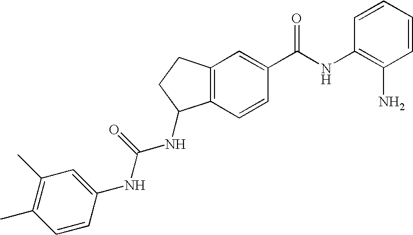 | N-(2-aminophenyl)-1-({[(3,4-dimethylphenyl)amino]carbonyl}amino)indane-5- carboxamide; | RT: 2.74 M + 1 = 415.1 |
| 119 | 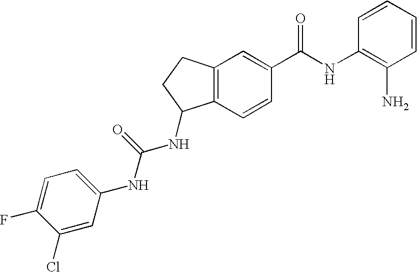 | N-(2-aminophenyl)-1-({[(3-chloro-4-fluorophenyl)amino]carbonyl}amino)indane-5- carboxamide; | RT: 2.83 M + 1 = 439.0 |
| 120 | 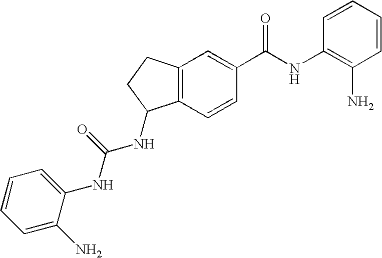 | N-(2-aminophenyl)-1-({[(2-aminophenyl)amino]carbonyl}amino)indane-5- carboxamide | RT: 1.79 M + 1 = 402.0 |
| 121 | 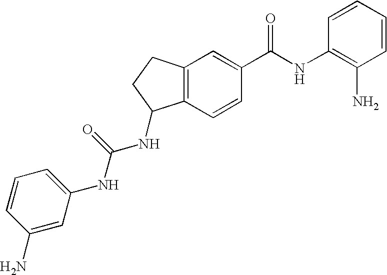 | N-(2-aminophenyl)-1-({[(3-aminophenyl)amino]carbonyl}amino)indane-5- carboxamide; | RT: 1.42 M + 1 = 402.0 |
| 122 | 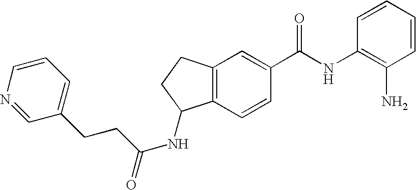 | N-(2-aminophenyl)-1-[(3-pyridin-3-ylpropanoyl)amino]indane-5-carboxamide; | RT: 1.20 M + 1 = 401.1 |
| 123 | 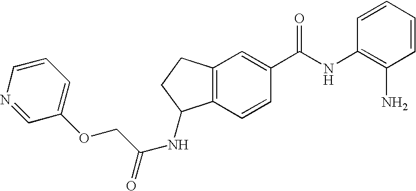 | N-(2-aminophenyl)-1-{[(pyridin-3-yloxy)acetyl]amino}indane-5-carboxamide; | RT: 1.34 M + 1 = 403.1 |

### B. PHYSIOLOGICAL ACTIVITY

### In Vitro Tumor Cell Proliferation Assay

The adherent tumor cell proliferation assay used to test the compounds of the present invention involves a readout called Cell Titre-Glo developed by Promega (Cunningham, B A “A Growing Issue: Cell Proliferation Assays. Modern kits ease quantification of cell growth” The Scientist 2001, 15(13), 26, and Crouch, S P et al., “The use of ATP bioluminescence as a measure of cell proliferation and cytotoxicity” Journal of Immunological Methods 1993, 160, 81-88).

HCT116 cells (colon carcinoma, purchased from ATCC) were plated in 96-well plates at 3000 cells/well in complete media with 10% Fetal Calf Serum and incubated 24 h at 37° C. Twenty-four h after plating, test compounds were added over a final concentration range of 10 nM to 20 μM in serial dilutions at a final DMSO concentration of 0.2%. Cells were incubated for 72 h at 37° C. in complete growth media after addition of the test compound. On day 4, using a Promega Cell Titer Glo Luminescent® assay kit, the cells are lysed and 100 microliters of substrate/buffer mixture is added to each well, mixed and incubated at room temperature for 8 min. The samples were read on a luminometer to measure the amount of ATP present in the cell lysates from each well, which corresponds to the number of viable cells in that well. Values read at 24 h incubation were subtracted as Day 0. For determination of IC50's, a linear regression analysis were used to determine drug concentration which results in a 50% inhibition of cell proliferation using this assay format.

Representative compounds of this invention showed a significant inhibition of tumor cell proliferation in the assays with HCT116 cells (>50% inhibition at 10 uM).

### C. OPERATIVE EXAMPLES RELATING TO PHARMACEUTICAL COMPOSITIONS

The compounds according to the invention can be converted into pharmaceutical preparations as follows:

### Tablet:

### Composition:

100 mg of the compound of Example 1, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg, diameter 8 mm, curvature radius 12 mm.

### Preparation:

The mixture of active component, lactose and starch is granulated with a 5% solution (m/m) of the PVP in water. After drying, the granules are mixed with magnesium stearate for 5 min. This mixture is moulded using a customary tablet press (tablet format, see above). The moulding force applied is typically 15 kN.

### Suspension for Oral Administration:

### Composition:

1000 mg of the compound of Example 1, 1000 mg of ethanol (96%), 400 mg of Rhodigel (xanthan gum from FMC, Pennsylvania, USA) and 99 g of water.

A single dose of 100 mg of the compound according to the invention is provided by 10 ml of oral suspension.

### Preparation:

The Rhodigel is suspended in ethanol and the active component is added to the suspension. The water is added with stirring. Stirring is continued for about 6 h until the swelling of the Rhodigel is complete.

1. A compound of formula (I) 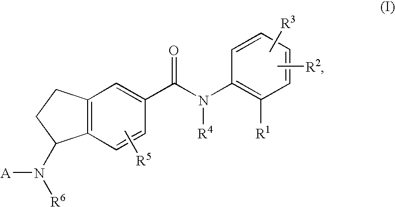
wherein
A represents 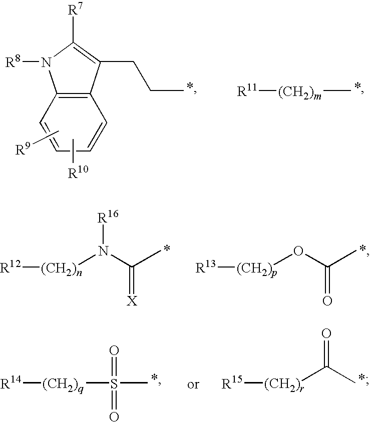
m, n, p, q and r represent 0, 1, 2, or 3;
R¹ represents hydroxy, alkoxy, amino or alkylamino;
R² represents hydrogen, alkyl or halo;
R³ represents hydrogen, alkyl or halo;
R⁴ represents hydrogen or alkyl;
R⁵ represents hydrogen, alkyl or halo;
R⁶ represents hydrogen; or
R⁶ represents alkyl, wherein alkyl can be substituted with 0, 1 or 2 substituents selected from the group consisting of halo, hydroxy, alkoxy, amino and alkylamino; or
R⁶ represents alkylcarbonyl; or
R⁶ represents alkylaminocarbonyl; or
R⁶ represents alkylsulfonyl;
R⁷ represents hydrogen or alkyl;
R⁸ represents hydrogen or alkyl;
R⁹ represents hydrogen, alkyl, halo, hydroxy or alkoxy;
R¹⁰ represents hydrogen, alkyl, halo, hydroxy or alkoxy;
R¹¹ represents hydrogen, phenyl, or benzthiazolyl;
R¹² represents pyridyl, thiazolyl, or indolyl optionally substituted with 1 or 2 substituents independently selected from the group consisting of alkyl, alkoxy and halo; or
R¹² represents phenyl optionally substituted with 1 or 2 substituents independently selected from the group consisting of alkyl, alkoxy, halo and amino;
R¹³ represents pyridyl or phenyl optionally substituted with 1 or 2 substituents independently selected from the group consisting of alkyl, alkoxy and halo;
R¹⁴ represents alkyl or phenyl optionally substituted with 1 or 2 substituents independently selected from the group consisting of alkyl, alkoxy and halo;
R¹⁵ represents hydrogen, pyridyl, pyridyloxy, phenoxy, or phenyl optionally substituted with 1 or 2 substituents independently selected from the group consisting of alkyl, alkoxy and halo;
R¹⁶ represents hydrogen or alkyl;
X represents oxygen or sulfur;
or a pharmaceutically acceptable salt thereof.

2. The compound of claim 1, wherein A represents

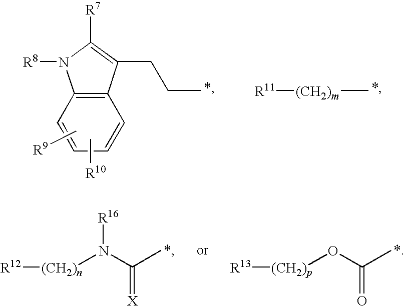

3. The compound of claim 1, wherein
R¹ represents hydroxy or amino;
R² represents hydrogen;
R³ represents hydrogen;
R⁴ represents hydrogen;
R⁵ represents hydrogen;
R⁶ represents hydrogen; or
R⁶ represents alkyl.

4. The compound of claim 1 for the treatment of disorders.

5. A pharmaceutical composition comprising a compound of claim 1.

6. The pharmaceutical composition of claim 5 in combination with at least one pharmaceutically acceptable, pharmaceutically safe carrier or excipient.

7. A process for preparing a pharmaceutical composition according to claim 6, comprising combining at least one compound according to claim 1 with at least one pharmaceutically acceptable, pharmaceutically safe carrier or excipient, mixing the combination and bringing the combination into a suitable administration form.

8. A use of a compound of claim 1 for manufacturing a pharmaceutical composition for the treatment of hyper-proliferative disorders.

9. A pharmaceutical composition according to claim 5 for the treatment of hyper-proliferative disorders.

10. A method of treating a disease or condition in a mammal, comprising administering to a mammal in need thereof an effective amount of a compound of claim 1.

11. A method of treating hyper-proliferative disorders in a mammal, comprising administering to a mammal in need thereof an effective amount of a compound of claim 1.
